(19) United States
(12) Patent Application Publication
(10) Pub. No.: US 2005/0107831 A1
(43) Pub. Date: May 19, 2005

(54) System for therapeutic application of energy

(75) Inventors: Guy Hill, Rock Spring, GA (US); Michael Moore, Rising Fawn, GA (US); David Bley, Chattanooga, TN (US); Jeff Pohl, Soddy Daisy, TN (US); Edward Dunlay, Harrison, TN (US)

Correspondence Address: David J. Hill;Chambliss, Bahner & Stophel, P.C., Two Union Square, 1000 Tallan Building, Chattanooga, TN, 37402, US

(73) Assignee: Encore Medical Asset Corporation

(21) Appl. No.: 10/990,668

(22) Filed: Nov. 17, 2004

Related U.S. Application Data
(60) Provisional application No. 60/520,994, filed on Nov. 18, 2003.

(51) Int. Cl. A61N 1/00

(52) U.S. Cl. 607/2

(57) ABSTRACT

A system for the therapeutic application of energy to a patient includes a primary unit that has a first generator for generating a first form of energy for therapeutic application to a patient. The primary unit also has a first outlet jack that is operatively connected to the first generator and adapted to receive a first therapeutic applicator, and a module receiver for receiving an upgrade module for enhancing the operation of the system. The system also includes a first therapeutic applicator for use in applying the first form of energy to a patient. The first therapeutic applicator is adapted to be operatively connected to the first generator through the first outlet jack. A preferred embodiment of the invention includes a first generator protocol operational mechanism that is operatively connected to the first generator and adapted to configure the operational parameters of the first generator. This embodiment of the invention also includes an interactive ailment-protocol selection interface that is operatively connected to the first generator protocol operational mechanism. Another embodiment of the invention includes a patient data storage device for storing identification data representative of at least one patient and a corresponding set of therapy information associated with each such patient. This embodiment also includes a display screen that is operatively connected to the patient data storage device and adapted to display therapy information and identification data contained in such patient data storage device, and a patient data input device that is operatively connected to the patient data storage device and the screen display and adapted to input therapy information and identification data for each such patient.

### CROSS REFERENCE TO RELATED APPLICATION

This application claims the benefit of U.S. Provisional Application No. 60/520,994, which was filed on Nov. 18, 2003.

### FIELD OF THE INVENTION

The invention relates generally to devices for use in applying energy to a human patient for treatment of various ailments and conditions. More particularly, the invention relates to a physical therapy device for use in treating a patient. In a preferred embodiment of the invention, the device may be used for the therapeutic application of energy to a patient in the form of electrical stimulation, ultrasound, radio frequency (or short wave) diathermy, eddy current heat and/or laser energy.

### BACKGROUND OF THE INVENTION

In recent years, a number of systems have been developed for applying a non-invasive therapeutic treatment to a patient to treat various ailments and conditions. These systems may operate by applying therapeutic ultrasound treatments, electrical stimulation or a combination of ultrasound treatments and electrical stimulation. Other systems may operate by applying electromagnetic radiation at various frequencies (including low level laser energy) to an injured or afflicted body part for therapeutic treatment.

A therapeutic ultrasound device typically employs a high-frequency oscillator and a power amplifier to generate a high frequency electrical signal that is delivered to a piezoelectric transducer housed in a handheld applicator. The transducer converts the electrical signal to inaudible high-frequency acoustic vibrations at the same frequency. This ultrasonic energy is then transmitted to the patient by applying a radiating plate on the transducer against the patient's skin. The effect of this application may produce diathermy or core tissue heating, or it may produce non-thermal therapeutic benefits. Operating in a similar manner, an electrical stimulation device typically employs a high frequency oscillator and a power amplifier to generate a high frequency electrical current that is then delivered to the patient through a pair of electrodes or through a probe.

Electromagnetic radiation in the infrared, visible and ultraviolet ranges may also be applied to tissues for therapeutic effect. Although high-energy laser radiation is commonly used as a surgical tool for cutting, cauterizing and ablating biological tissue by application of concentrated heat energy, low levels of electromagnetic radiation may have a non-thermal, biostimulative effect on biological tissues. For example, the therapeutic application of low level laser therapy (“LLLT”) produces beneficial clinical effects in the treatment of musculoskeletal, neurological and soft tissue conditions. Like ultrasound therapy and electrical stimulation, LLLT is non-invasive and avoids the potential side effects of drug therapy. More specifically, it is believed that LLLT delivers photons to targeted tissues, penetrating layers of skin to reach internal tissues to produce a specific, non-thermal photochemical effect at the cellular level. One known effect of LLLT is to enhance microcirculation of both blood and lymph. Generally, electromagnetic radiation (including laser energy) is delivered for therapeutic effect by means of a handheld probe. The probe includes one or more laser or LED diodes, each of which is adapted to emit a beam of electromagnetic radiation. Preferably, the probe includes a plurality of diodes that are arranged so that the beams emitted therefrom intersect a short distance from the head of the probe. A control unit associated with the probe includes circuitry for controlling the amount of energy that is delivered to the diodes. U.S. Pat. No. 6,214,035, No. 6,267,780, No. 6,273,905 and No. 6,312,451 describe various methods and devices for providing LLLT.

Various forms of therapeutic diathermy may be employed to treat ailments or conditions in which warming of core body tissues is effective. As mentioned above, ultrasound diathermy involves the application of inaudible, high-frequency acoustic vibrations to tissues. Microwave diathermy involves the application of microwave energy to tissues to generate an electrical field with relatively low magnetic-field energy, which thereby induces heat by intra-molecular vibration of highly polarized molecules within the tissues. Radio frequency diathermy employs short wave radio frequency electromagnetic fields to induce thermal effects derived from high frequency molecular vibrations. The application of radio frequency electromagnetic radiation may also have non-thermal effects. Both radio frequency and microwave electromagnetic radiation may be applied for therapeutic purposes through coils contained in a garment or wrap that is applied to the tissue to be treated.

A number of different factors can affect the proper therapeutic application of energy in the form of ultrasound, electromagnetic radiation and electrical current to tissue, including the nature of the condition or ailment to be treated, the area and depth of the tissue to be treated, the size of the transducer, electrodes, probe or other therapeutic applicator employed, the amount of energy applied, the frequency, amplitude and specific type of wave form produced by an electrotherapeutic oscillator (or the wavelength of the laser or other type of electromagnetic radiation produced or the ultrasound pulse duration produced), and the time for a treatment session. Because different injuries and conditions require different treatment techniques, and because each treatment technique has numerous variations in at least some of the factors described above, there are many treatment options. In order to provide the best rehabilitation or other therapeutic treatment, the physical therapist or other health care professional must have a reference available which describes the various clinical protocols and application procedures for application of electrical stimulation, therapeutic ultrasound, microwave or short wave radio frequency diathermy or therapeutic laser energy for the various physical ailments and conditions. Furthermore, the practitioner must have some means for configuring the equipment according to the appropriate protocol for the condition to be treated. U.S. Pat. No. 5,578,060, the disclosure of which is incorporated herein by reference, describes a physical therapy apparatus for therapeutic application of therapeutic ultrasound or electrical stimulation. This apparatus includes a memory device for storing identification data representative of a plurality of physical ailments for each of a plurality of human body areas and a set of operational parameters associated with each such physical ailment and each such body area. The device also includes an ailment display screen for displaying identification data representative of a plurality of physical ailments which are associated with the human body areas and an ailment selector to permit the operator to select an ailment associated with a particular body area. Upon selection by an operator of a particular body area and ailment, the apparatus will obtain the appropriate operational parameters from the memory device and configure the output device to provide therapeutic ultrasound or electrical stimulation to the identified body part according to the stored operational parameters.

It is known to monitor muscle activation or contraction using an electromyograph (EMG) device to help a therapist isolate the muscles that need treatment during a therapy session. EMG is commonly used in orthopedic, sports medicine, neurological rehabilitation and incontinence therapy. It is also known to measure joint articulation and the force or pressure generated by muscle contraction during or in association with a treatment session. Typically, the devices for making such measurements are not integrated into a therapeutic treatment device.

It would be desirable if a therapeutic treatment device could be provided that could be readily configured to provide any of various treatment options, including electrical stimulation, therapeutic ultrasound and therapeutic laser energy. It would also be desirable if such a device could be provided with anatomical graphics that may be displayed on a screen for ease in identifying the body part to be treated. It would also be desirable if such a device could be provided with enhanced clinical protocols that may be modified for a particular patient or situation. It would also be desirable if such a device could be provided with modular components for enhancing its operational flexibility and operating parameters. It would also be desirable if such a device could be provided with a system for recording treatment parameters and treatment session details for a particular patient and for storing such information for subsequent recall. Further, it would be desirable if such an information storage system could be configured for portability so that the information for each patient could be stored on an electronic data card which could be placed in the patient's file. It would also be desirable if such a portable storage system could be used to transfer information to a personal computer for use in generating a report of treatment history.

### ADVANTAGES OF THE INVENTION

Among the advantages of a preferred embodiment of the invention is a system of compatible modular devices that may be readily connected to enhance the operation of the system. Some of the preferred modules may provide various treatment options, including (but not limited to) electrical stimulation, therapeutic ultrasound and therapeutic laser treatment. Some such modules may enhance the operational flexibility of the main unit, such as by providing a rechargeable battery module, and some may enhance the operating parameters of the main unit, such as by providing a module to increase the number of channels of stimulation that can be applied or an EMG module that may be employed to set a patient's EMG level to activate muscle stimulation. Another advantage of a preferred embodiment of the invention is a display device that may display anatomical drawings for ease in identifying the body part to be treated. Yet another advantage of a preferred embodiment of the invention is enhanced clinical protocols that may be modified for a particular patient or situation. Still another advantage of a preferred embodiment of the invention is a system for recording treatment parameters and treatment session details for a particular patient and for storing such information for recall. Another advantage of this embodiment of the invention is a portable information storage system that stores information for each patient on an electronic data card which can be placed in the patient's file or used in connection with a computer-compatible card read/write device to transfer information to a personal computer for generating a report of treatment history.

Additional advantages of the invention will become apparent from an examination of the drawings and the ensuing description.

### EXPLANATION OF TECHNICAL TERMS

As used herein, the term “therapeutic applicator” refers to a device by or through which a therapeutic dosage of energy is delivered to a patient. Therapeutic applicators include (but are not limited to) electrodes, electrode pairs, probes, transducers and wraps or garments containing coils.

As used herein, the term “diathermy” refers to a type of therapy in which body tissues are heated by the resistance of body tissues to energy delivered to or applied to such tissues. Diathermy may employ energy sources such as high-frequency acoustic vibrations (ultrasound energy), microwaves and radio frequency (short wave) electromagnetic radiation.

As used herein, the terms “electromyography”, “EMG” and similar terms refer to methods and devices for detecting and/or measuring the extracellular activity of skeletal muscles.

As used herein, the terms “electrotherapy”, “electrical stimulation”, “e-stim” and similar terms refer to any of various methods and devices for treatment of disease or injury by a therapeutic application of electrical current or voltage.

As used herein, the terms “low level laser therapy”, “LLLT” and similar terms refer to the therapeutic application of low levels of laser energy to a patient.

As used herein, the term “outlet jack” and similar terms refers to a connector, connectors, receptacle or receptacles by or through which a therapeutic applicator may be operatively connected.

As used herein, the term “ultrasound” and similar terms refer to a type of therapy employing high-frequency acoustic vibrations.

### SUMMARY OF THE INVENTION

The invention comprises a system for the therapeutic application of energy to a patient, which system includes a primary unit. The primary unit includes a first generator for generating a first form of energy for therapeutic application to a patient, a first outlet jack that is operatively connected to the first generator and adapted to receive a first therapeutic applicator, and a first therapeutic applicator for use in applying the first form of energy to a patient, which applicator is adapted to be operatively connected to the first generator through the first outlet jack. The primary unit also includes a module receiver for receiving an upgrade module for enhancing the operation of the system.

The preferred embodiment of the invention comprises a primary electrotherapy unit which provides for treatment using two channels of electrical stimulation and one ultrasound output. This primary unit includes a plurality of clinical waveforms and a fully functional 1 MHz and 3 MHz ultrasound component. Ten of the waveforms that may be produced by the main unit for electrical stimulation are FDA approved for electrical stimulation, namely the TENS asymmetrical biphasic, TENS symmetrical biphasic, direct current, monophasic high volt (either positive or negative), interferential (IFC) four pole (traditional), interferential (IFC) two pole (premodulated), VMS™, VMS™ Burst, Russian and Microcurrent (positive, negative or alternating) waveforms. Other waveforms that may be produced by the primary unit for electrical stimulation include TENS alternating rectangular, TENS monophasic rectangular, Diadynamic, monophasic rectangular pulsed, monophasic triangular pulsed, galvanic continuous, galvanic interrupted, Träbert (Ultrareiz), surged monophasic rectangular and surged monophasic triangular waveforms.

Preferably, the primary unit includes a display screen and associated software to display graphics and textual information about the various conditions and ailments that may be treated, as well as detailed treatment protocols for each such condition and ailment, which protocols may be modified for a particular patient or condition. The main unit is preferably provided with one or more on-board data storage devices and an on-board multimedia card reader which, in cooperation with the color or monochrome display screen, allow the therapist and the patient to view vivid graphical anatomical or pathological libraries on the primary unit. The anatomical graphics displayed are preferably specific to particular body parts or areas and tissue types. The on-board multimedia card reader may also be employed to permit upgrading of the operating software of the primary unit to functionally change the performance of such unit.

The preferred primary electrotherapy unit also accommodates various modules for enhancing the operation or performance of the system such as by providing additional treatment options. Additional modules that may be provided include a module for adding two additional channels of electrical stimulation, EMG modules including a surface EMG (sEMG) module and a surface EMG plus electrical stimulation (sEMG+stim) module, a laser energy (or other type of electromagnetic radiation) module (or more specifically, a radio frequency or microwave diathermy module), a module for delivering electrical current through a probe (for incontinence therapy) a battery module and a vacuum electrode module to accommodate vacuum-type electrodes.

The preferred primary electrotherapy unit also accommodates serial devices such as a Hand Dynamometry device that is used to determine hand strength, an Electrogoniometry device that is used to measure joint articulation, and a pressure pad transducer (e.g. a multi-purpose dynamometer) that is used to measure force of muscular contraction.

The preferred embodiment of the invention also includes a system for recording treatment parameters and treatment session details for a particular patient and for storing such information for recall. This embodiment provides for storage of such information for each patient on an electronic data card which can be placed in the patient's file or used in connection with a separate stand-alone USB card reader to transfer information to a personal computer for generating a report of treatment history. Software may also be provided for a personal computer that is used with the USB card reader that will permit treatment session notes and other information to be recorded on the electronic data card, which additional information may be later retrieved for viewing on the data screen of the primary unit.

In order to facilitate an understanding of the invention, the preferred embodiments of the invention are illustrated in the drawings, and a detailed description thereof follows. It is not intended, however, that the invention be limited to the particular embodiments described or to use in connection with the apparatus illustrated herein. Various modifications and alternative embodiments such as would ordinarily occur to one skilled in the art to which the invention relates are also contemplated and included within the scope of the invention described and claimed herein.

### BRIEF DESCRIPTION OF THE DRAWINGS

The presently preferred embodiments of the invention are illustrated in the accompanying drawings, in which:

FIG. 1 is a perspective view of a primary unit of the preferred embodiment of the invention.

FIG. 2 is a front view of a portion of the primary unit of FIG. 1, with the front access panel removed.

FIG. 3 is a left side view of the primary unit of FIG. 1.

FIG. 4 is a rear view of the primary unit of FIGS. 1-3.

FIG. 5 is a bottom view of the primary unit of FIGS. 1-4.

FIG. 6 is a perspective view illustrating a portion of a first embodiment of a module for enhancing the operation of the system which includes a primary unit such as is shown in FIGS. 1-5.

FIG. 7 is a front view of a second embodiment of a module for enhancing the operation of the system which includes a primary unit such as is shown in FIGS. 1-5.

FIG. 8 is a bottom view of either the first embodiment of the module illustrated in FIG. 6 or the second embodiment of the module illustrated in FIG. 7.

FIG. 9 is a perspective view of a primary unit such as is shown in FIGS. 1-5 to which is attached a module such as is shown in FIGS. 6 and 8 or a module of FIGS. 7-8.

FIG. 10 is a perspective view of a third embodiment of a module for enhancing the operation of the system which includes a primary unit such as is shown in FIGS. 1-5.

FIG. 11 is a bottom view of the module of FIG. 10.

FIG. 12 is an exploded perspective view of a preferred assembly of the invention, showing a primary unit such as is shown in FIGS. 1-5 to which is attached a module such as is shown in FIGS. 6 and 8 or 7-8 and a first EMG module of the type shown in FIGS. 10-11. FIG. 12 also shows a second EMG module of the type shown in FIGS. 10-11 attached to the module that is illustrated in FIGS. 7-8.

FIG. 13 is a flow chart showing preferred expansion capabilities for enhancing the performance of a primary unit such as is illustrated in FIGS. 1-5 by addition of modules such as are illustrated in FIGS. 6 and 8, FIGS. 7-8 and FIGS. 10-11.

FIG. 14 illustrates four types of therapeutic applicators for use in applying a form of energy to patient in connection with a preferred embodiment of the invention.

FIG. 15 illustrates the display screen and function keys of the preferred embodiment of the primary unit of the invention.

FIG. 16 illustrates a page that may be displayed on the display screen of the preferred embodiment of the invention when the operator has selected electrotherapy as the treatment modality.

FIG. 17A is a schematic block diagram illustrating the interactive ailment protocol selection interface of a preferred embodiment of the invention.

FIG. 17B is a schematic block diagram of the electrical stimulation data file of a memory device of the interactive ailment protocol selection interface of a preferred embodiment of the invention.

FIG. 17C is a schematic block diagram of the library data file of a memory device of the interactive ailment protocol selection interface of a preferred embodiment of the invention.

FIG. 18 is a flow chart illustrating the use of a preferred embodiment of the invention to select an appropriate treatment protocol for treatment of any of various conditions and ailments.

FIG. 19 illustrates a page that may be displayed on the display screen of the preferred embodiment of the invention when the operator has accessed the clinical protocols library.

FIG. 20 is a flow chart illustrating the use of an on-board multimedia card reader to view graphical anatomical and/or pathological libraries on a display screen for a selected body part or area.

FIG. 21A illustrates a first page that may be displayed on the display screen of the preferred embodiment of the invention when the operator has accessed the information from a multimedia card.

FIG. 21B illustrates a second page that may be displayed on the display screen of the preferred embodiment of the invention when the operator has accessed the information from a multimedia card.

FIG. 22 is a schematic block diagram illustrating the patient data management system interface of a preferred embodiment of the invention.

FIG. 23 illustrates an external patient data read/write device that is adapted to be operated in connection with a personal computer (shown schematically) in a preferred embodiment of the invention.

FIG. 24 is a flow chart illustrating the use of an external read/write device and personal computer in connection with a preferred embodiment of the invention.

FIG. 25 is a flow chart illustrating the use of the internal card reader component in operating a preferred embodiment of the invention using a patient data card.

### DESCRIPTION OF THE PREFERRED EMBODIMENTS OF THE INVENTION

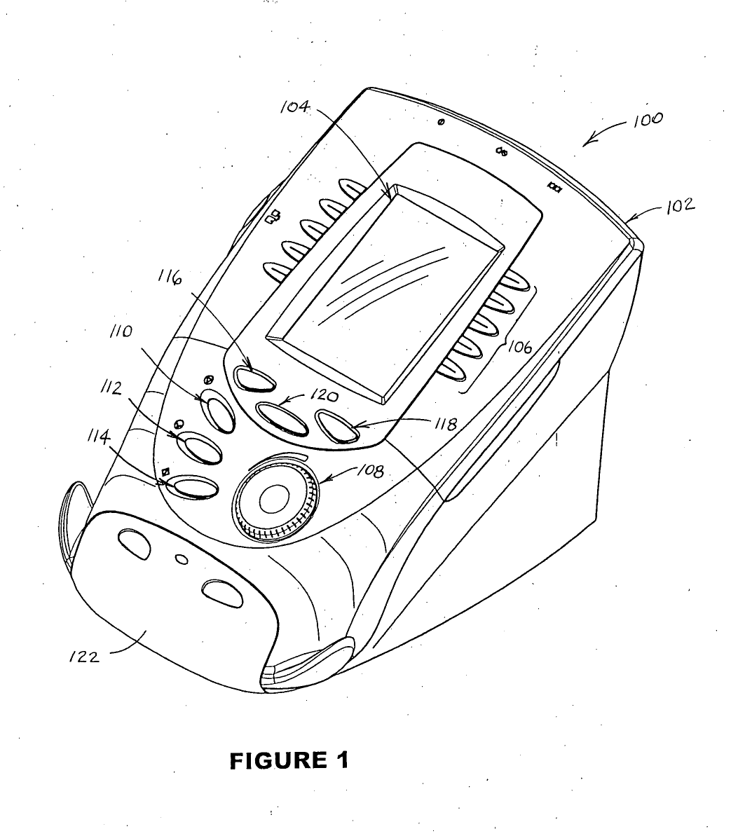

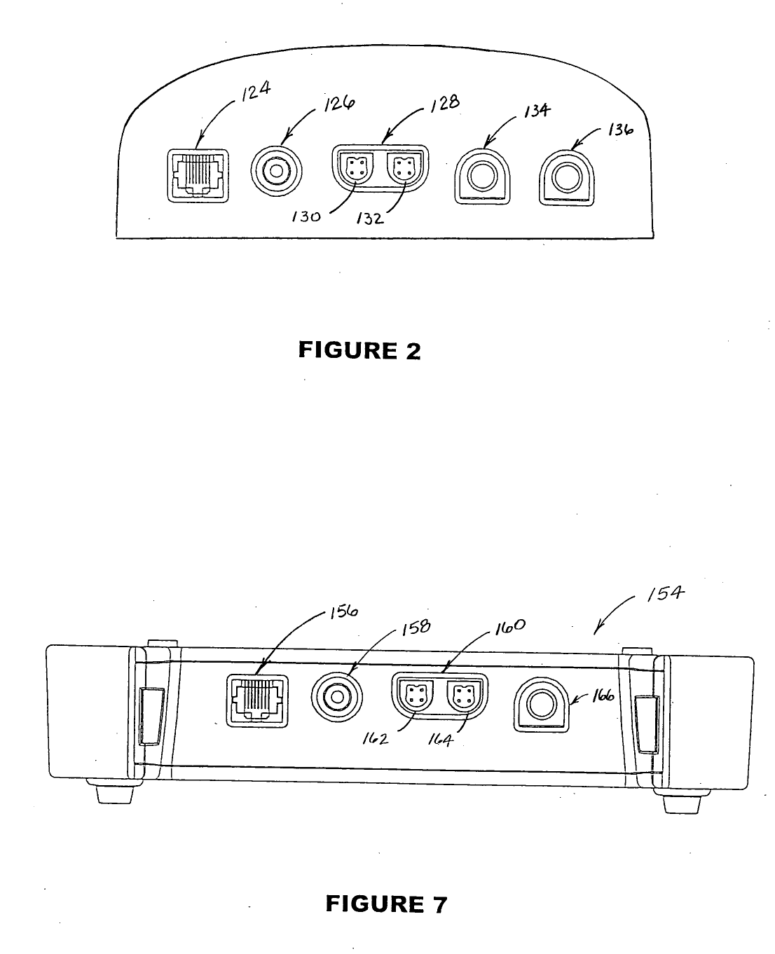

Referring now to the drawings, a preferred embodiment of the invention is illustrated by primary unit 100, which comprises the main component of a modular system for providing the therapeutic application of energy in multiple forms. The embodiment shown in FIGS. 1-5 comprises a combination two-channel electrotherapy and ultrasound system. Primary unit 100 includes cabinet 102 having display screen 104 and a user interface in the form of function keys 106 (including keys on both sides of the display screen), which form a part of the interactive interface of the preferred system. Other function keys include knob 108 which provides therapy “Intensity Control” and button 110, which is a therapy session “Start” key. Button 112 is a therapy session “Pause” key and button 114 is a therapy session “Stop” key. Button 116 is a “Home Page” key and button 118 is a “Back” key (to return to the previous page displayed). Button 120 accesses the Clinical Resource Library database. Front access panel 122 provides access to a plurality of outlet jacks (shown in FIG. 2), including operator remote control connector 124, patient interrupt switch connector 126, electrical stimulation outlet jack 128 (comprised of channel A connector 130 and channel B connector 132), microcurrent probe connector 134 (for use when electrical stimulation is applied using a microcurrent waveform) and ultrasound applicator connector 136.

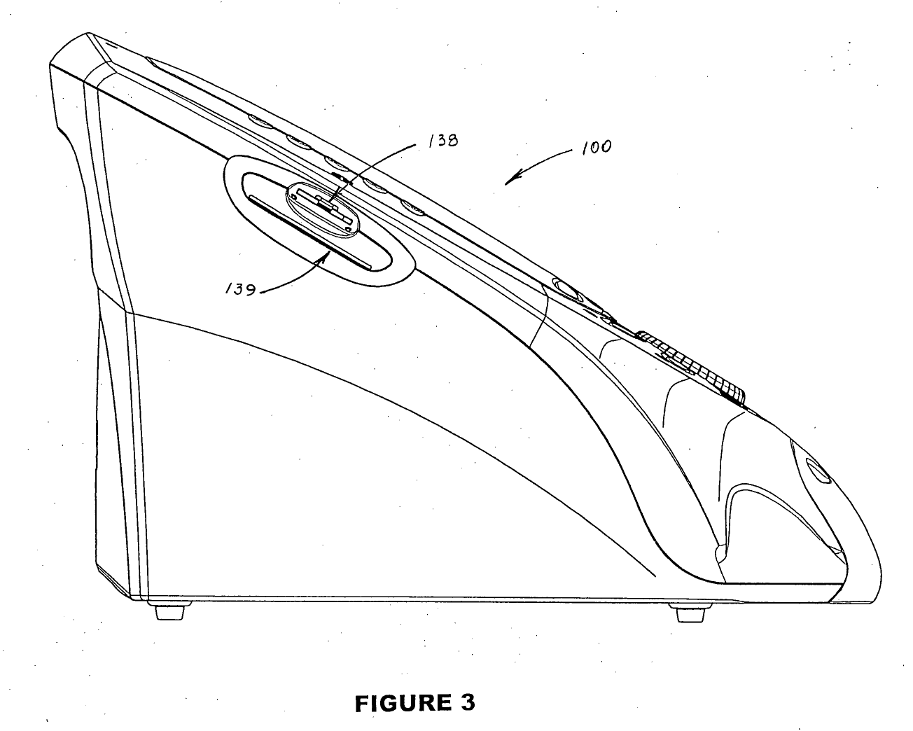

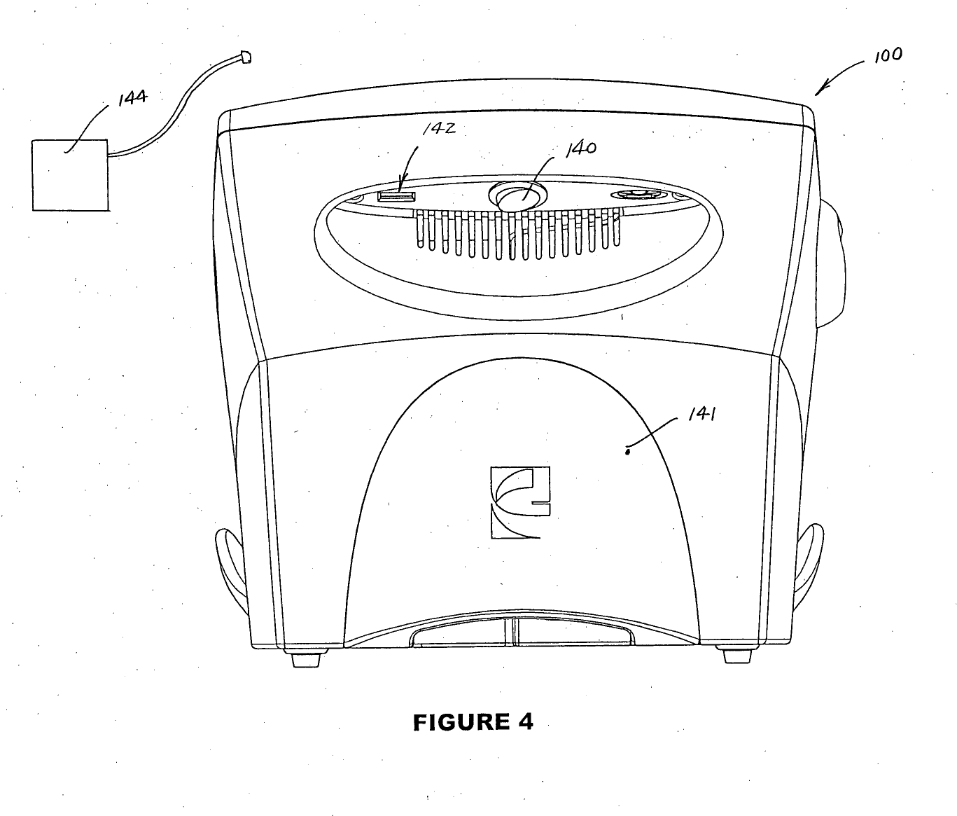

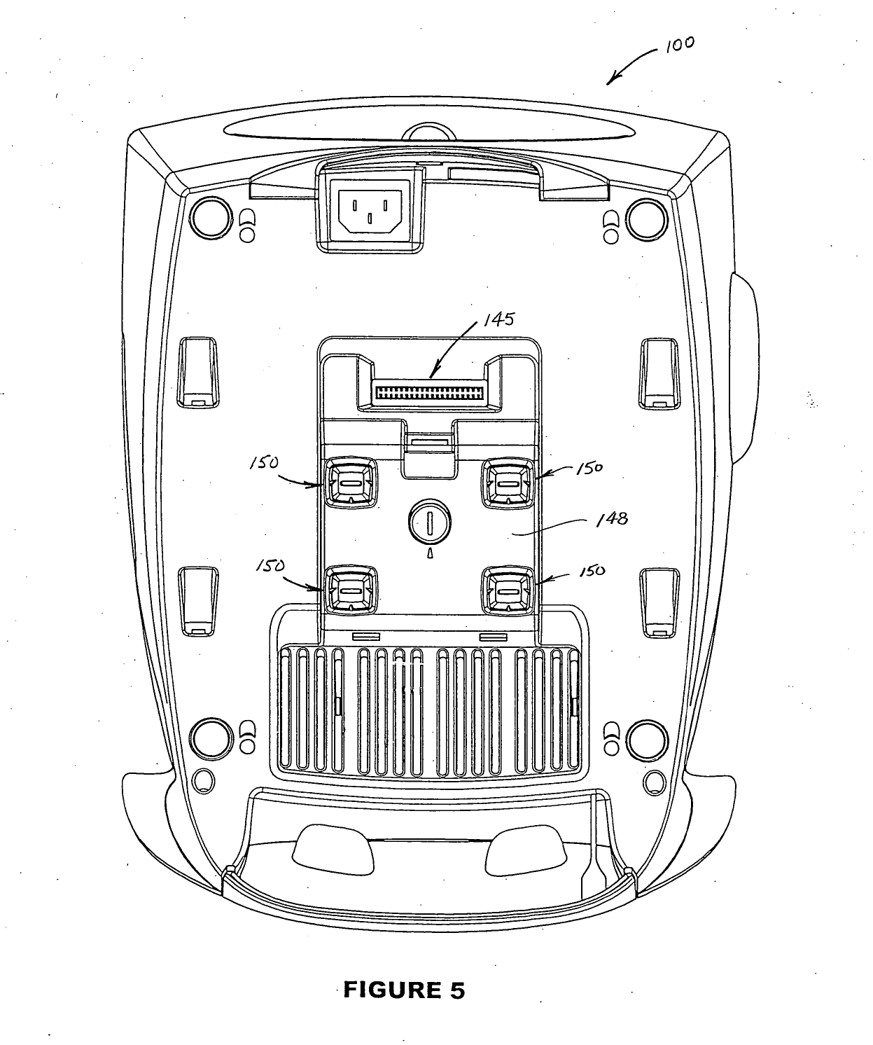

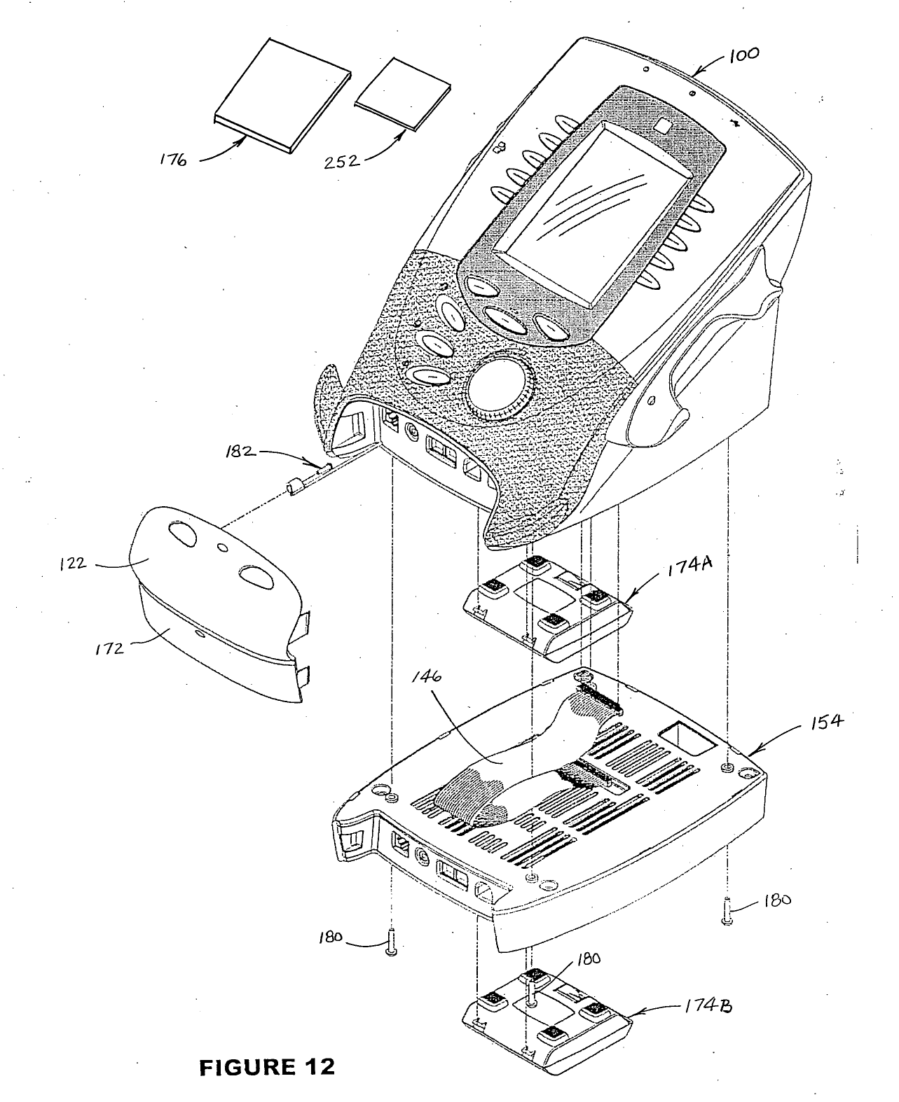

FIG. 3 shows the left side of preferred primary unit 100 illustrating multimedia card access port 138 and patient data card access port 139. FIG. 4 shows the rear of preferred primary unit 100 illustrating system power switch 140 and rear access panel 141, behind which is located the main power cord (not shown). FIG. 4 also shows the location of serial connector outlet jack 143 for connection of a serial device 144 (shown schematically) such as a hand dynamometry device, an electrogoniometry device or a pressure pad transducer. FIG. 5 shows the bottom of primary unit 100, including a first module receiver which is adapted to receive several types of upgrade modules for enhancing the operation of the system. This first module receiver comprises ribbon cable connector 145 and an associated ribbon cable 146 (shown in FIG. 12). The first module receiver is adapted to receive an upgrade module such as a rechargeable battery module, a two-channel electrical stimulation upgrade module, a vacuum electrode module (which is adapted to permit the application of electrical stimulation using vacuum electrodes instead of adhesively applied electrodes) or a module that is adapted to generate energy in the form of electromagnetic radiation (such as an LLLT module). Second module receiver 148, which includes PC board contacts 150, is adapted to receive another type of upgrade module for enhancing the operation of the system, such as an EMG module (described in more detail hereinafter).

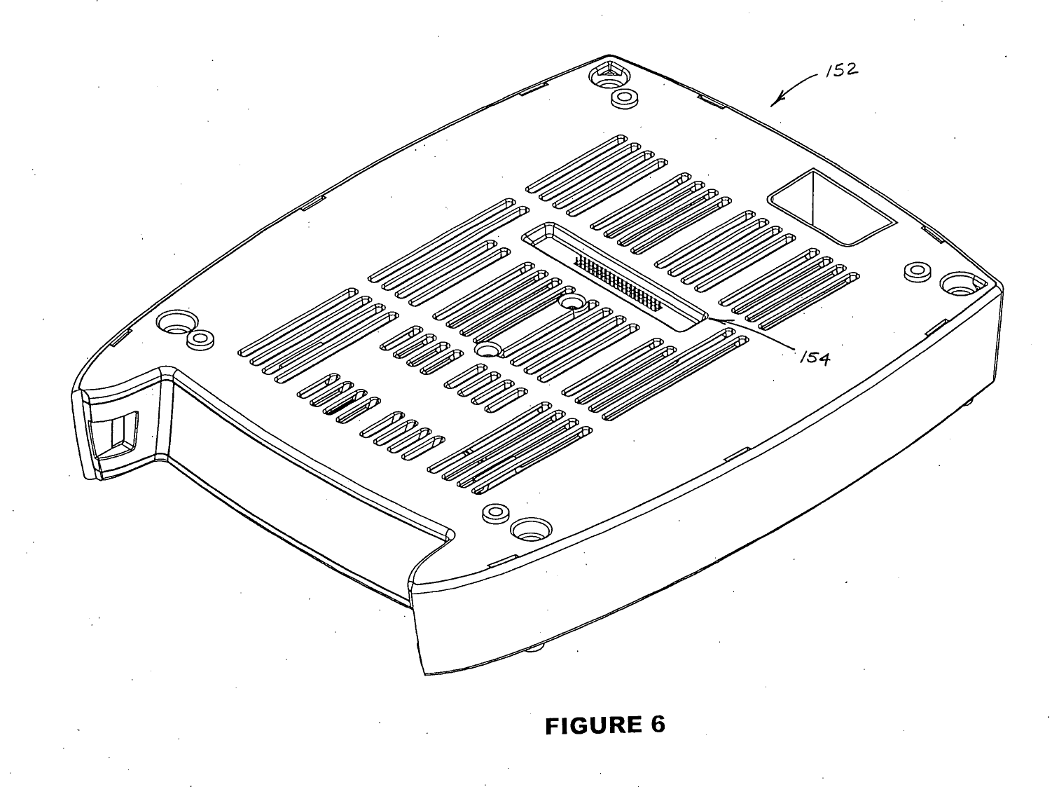

FIG. 6 is an upper perspective view of battery module 152, a first embodiment of an upgrade module for enhancing the operation of the system. Preferably, module 152 comprises a rechargeable NiMH battery which is adapted to be operatively connected to a first generator (described in more detail hereinafter) of primary unit 100 for generating a first form of energy for therapeutic application to a patient. Module 152 includes ribbon cable connector 154 for connection of module 152 to the first module receiver of primary unit 100.

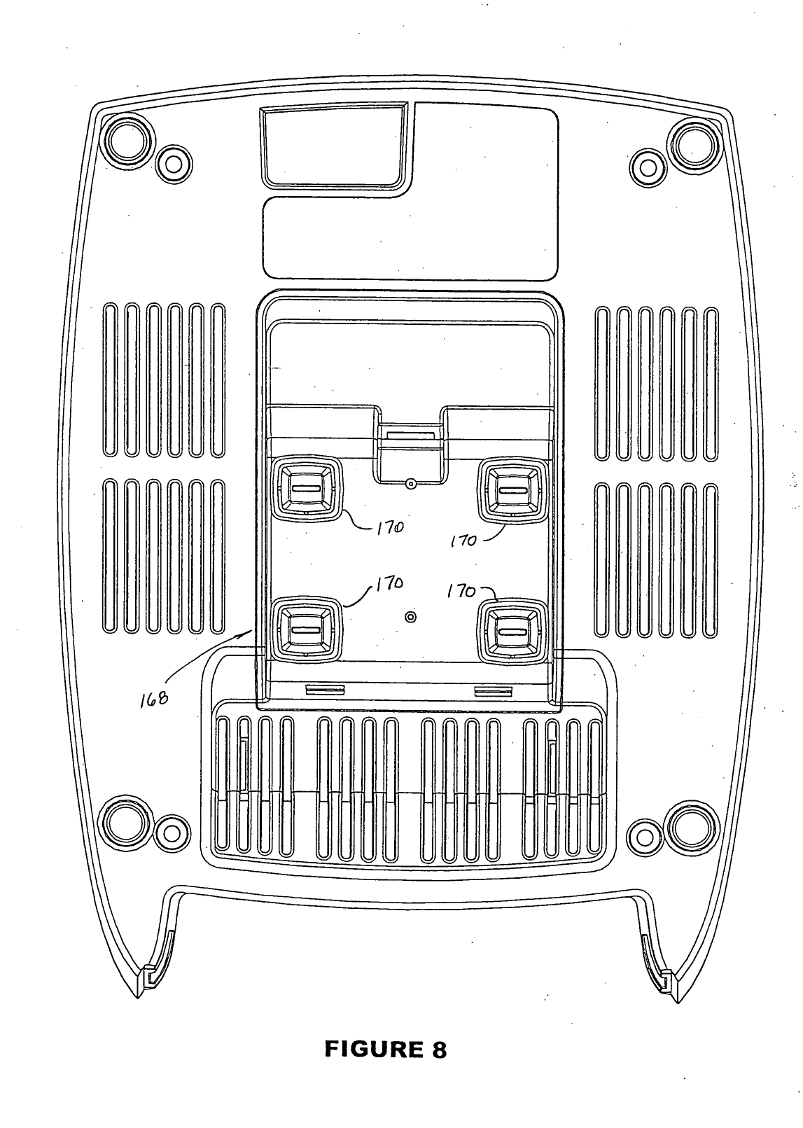

FIG. 7 illustrates a front view of a second embodiment of an upgrade module for enhancing the operation of the system. Module 154 is preferably identical to module 152 in shape, and both module 152 and module 154 have essentially identical upper and lower surfaces. Module 154 may comprise a two-channel electrical stimulation upgrade module, a vacuum electrode module (which is adapted to permit the application of electrical stimulation using vacuum electrodes instead of adhesively applied electrodes) or a module that is adapted to generate energy in the form of electromagnetic radiation (such as an LLLT module). Module 154 includes ribbon cable connector 156 for connection of module 154 to the first module receiver of primary unit 100. Also as shown in FIG. 7, module 154 includes a plurality of outlet jacks, such as operator remote control connector 156 (similar to remote control connector 124 of primary unit 100), patient interrupt switch connector 158 (similar to interrupt switch connector 126 of primary unit 100), electrical stimulation outlet jack 160 (comprised of channel C connector 162 and channel D connector 164) and microcurrent probe connector 166 (similar to microcurrent probe connector 134 of primary unit 100).

Although not shown in the drawings in connection with the preferred embodiment, it is also contemplated within the scope of the invention that the primary unit may comprise two-channel or four-channel electrical stimulation without an additional energy-form generator for generating an additional form of energy for therapeutic application. In such event, a module such as module 154 could be provided to add an additional energy-form generator (such as, for example, an ultrasound generator). In such case, the upgrade module could also include an ultrasound applicator connector similar to ultrasound applicator connector 136 of primary unit 100.

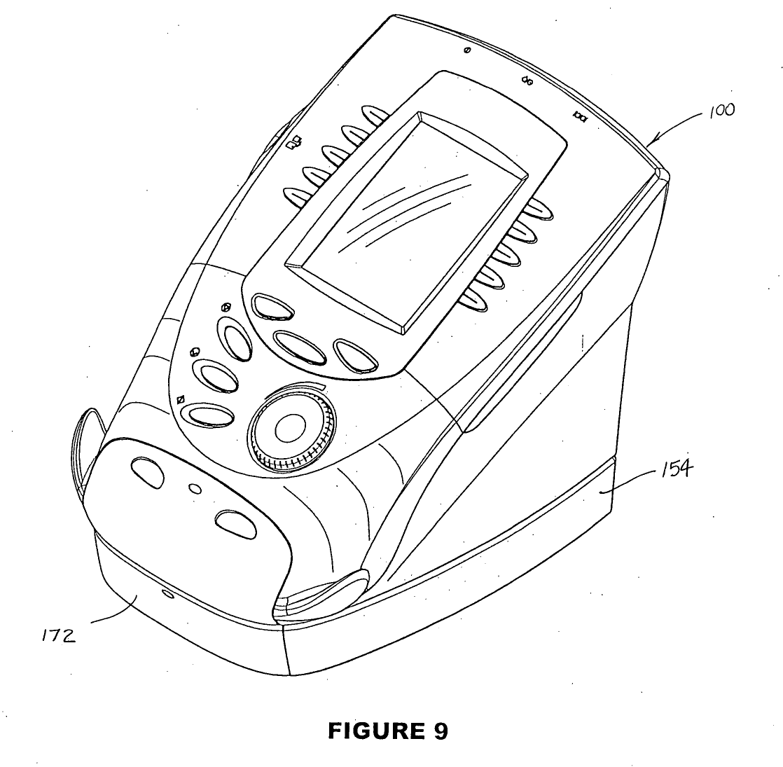

FIG. 8 illustrates a bottom view of either module 152 or module 154. Preferably, these modules include secondary module receiver 168 which is essentially identical to second module receiver 148 of the primary unit. Secondary module receiver 168, which includes PC board contacts 170, is adapted to receive another type of upgrade module for enhancing the operation of the system, such as an EMG module (described in more detail hereinafter). FIG. 9 illustrates the combination of primary unit 100 and an upgrade module such as module 154. A removable front module cover plate 172 is mounted on the front of module 154 to cover the outlet jacks.

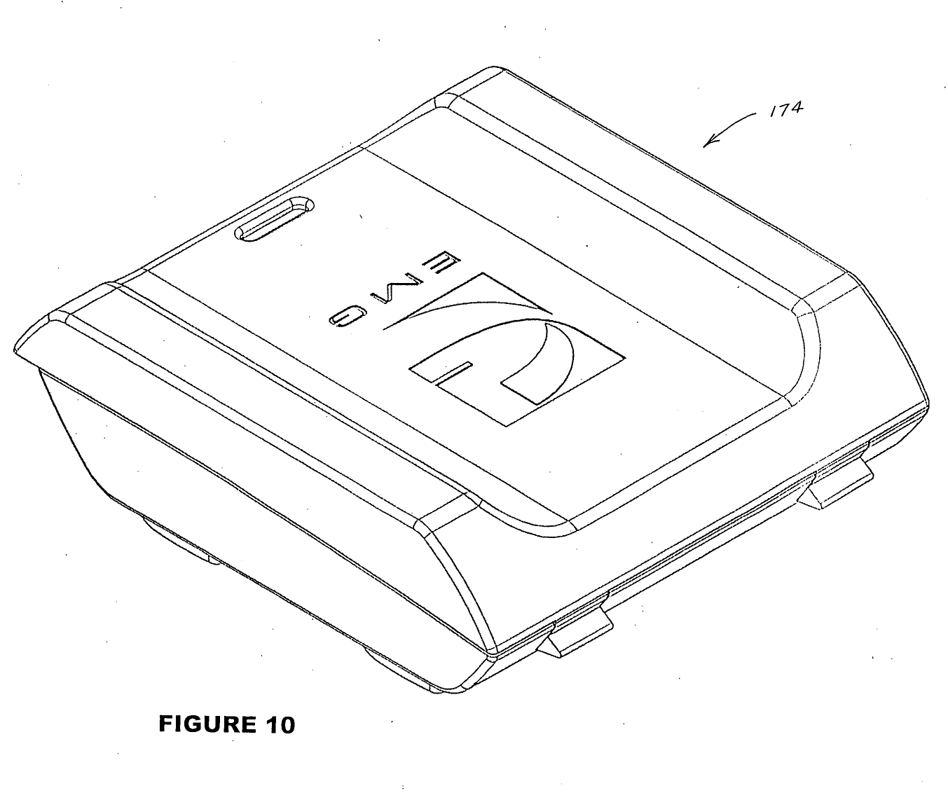

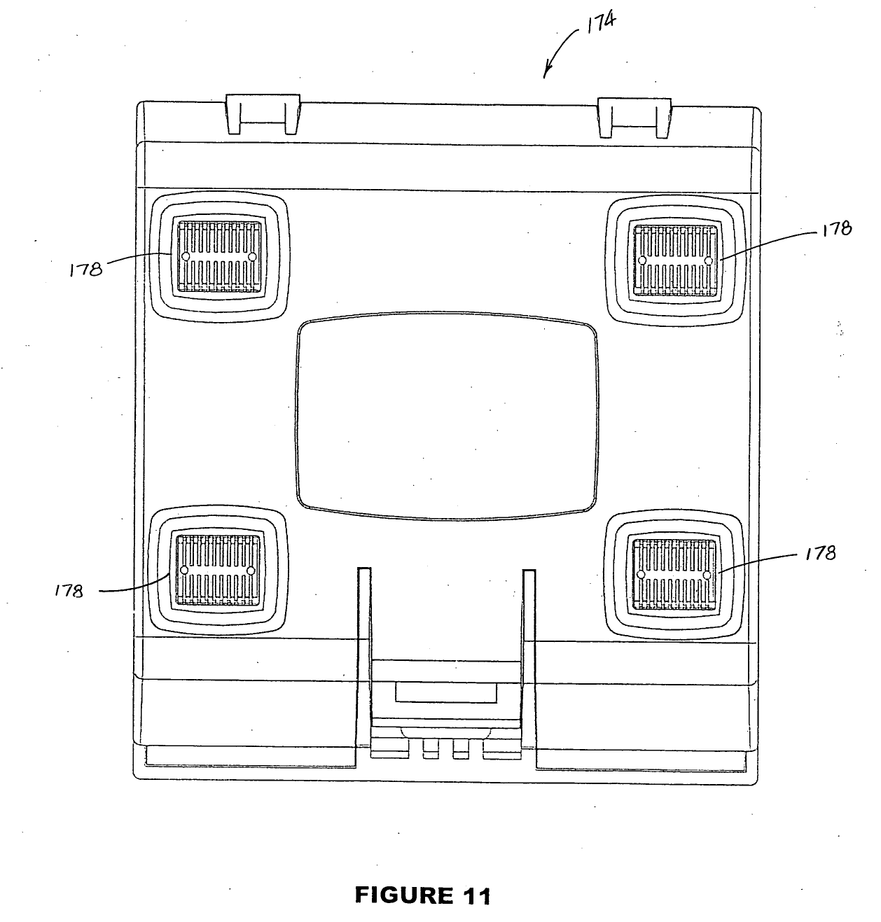

FIGS. 10 and 11 illustrate a second type of upgrade module that is adapted to be received by second module receiver 148 of primary unit 100, or by secondary module receiver 168 of upgrade module 154. Preferably upgrade module 174 is a surface EMG module which may be used with a pair of electrodes (not shown) connected to outlet jack 128 of the primary unit or outlet jack 160 of module 154 to permit monitoring or measurement of the extracellular activity of the skeletal muscles over which the electrodes are placed. The muscle activity may be shown in graphical form on display screen 104 or it may be stored on a patient data storage device such as removable patient data card 176 (shown in FIG. 12). One type of EMG module may also be used to permit a patient to initiate electrical stimulation by muscle contraction. EMG module 174 includes contacts 178 which are adapted to mate with PC board contacts 150 of second module receiver 148 of the primary unit or with PC board contacts 170 of secondary module receiver 168 of module 154. FIG. 12 shows a primary unit 100 having a second module receiver (shown in FIG. 5) to which an EMG module 174A is attached. Primary unit 100 also has a first module receiver which includes ribbon cable 146 to which upgrade module 154 is attached. Module 154 is secured to the bottom of primary unit 100 by a plurality of screws 180, and a second EMG module 174B is attached to the secondary module receiver (shown in FIG. 8) of module 154. In this example, the primary unit or the combination of the primary unit and the upgrade module comprise four channels of electrical stimulation. Electrical stimulation channels A and B are provided with EMG capability by EMG module 174A, and channels C and D are provided with EMG capability by EMG module 174B. Front covers 122 and 172 are joined together and attached to the primary unit by lanyard 182.

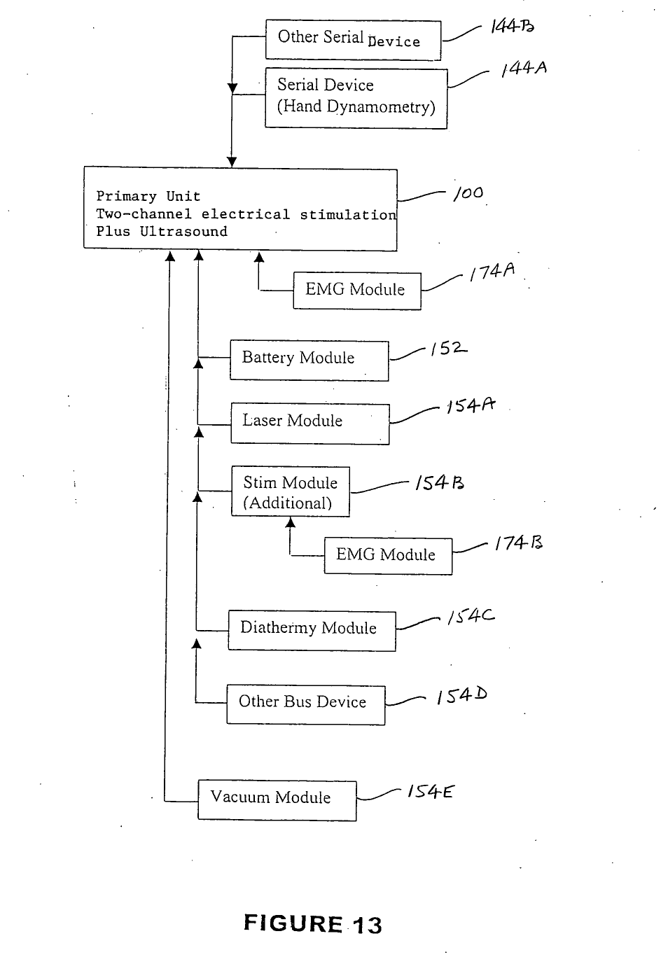

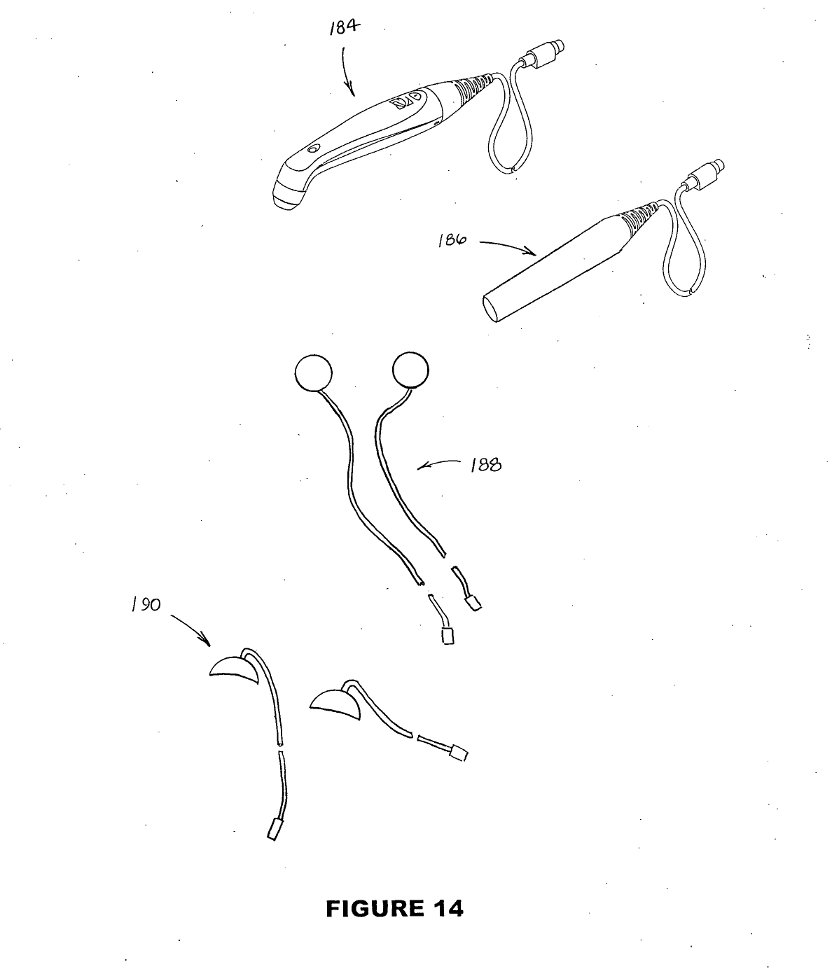

FIG. 13 illustrates the modular expansion capabilities of the invention. As shown therein, primary unit 100 comprises a combination two-channel electrotherapy and ultrasound system, including electrical stimulation capability through two channels. This preferred primary unit will include a first generator for generating a first form of energy, namely electrical current (comprising two channels of electrical stimulation), for therapeutic application to a patient. This preferred primary unit will also include a second generator for generating a second form of energy, namely high-frequency acoustic vibrations (or ultrasound energy) for therapeutic application to a patient. As shown in FIG. 13, primary unit 100 may have a hand dynamometry serial device 144A or another serial device 144B attached to it through serial port 142 (shown in FIG. 4), and in addition, it may have EMG module 174A attached through second module receiver 148 (shown in FIG. 5). Primary unit 100 may also have attached to it one or more upgrade modules through its first module receiver and/or through the secondary modules of the upgrade modules. Thus, for example, primary unit 100 may be attached, through its first module receiver, to battery module 152 for powering the system and/or to vacuum module 154D to permit electrical stimulation to be delivered to a patient using vacuum electrodes instead of adhesive electrodes. Primary unit 100 may also be connected to an upgrade module which includes a generator for generating a form of energy for therapeutic application to a patient. Thus for example, primary unit 100 may be connected to laser module 154A which includes an energy-form generator for generating LLLT, or to channel upgrade module 154B which includes an energy-form generator for generating two additional channels of electrical stimulation, or to radio frequency diathermy module 154C which includes an energy-form generator for generating electromagnetic radiation within the radio frequency range, or to another upgrade module 154C which includes an energy-form generator for generating another form of energy for therapeutic application. In addition, if a channel upgrade module 154B is attached to the primary unit, a second EMG module 174B may be attached to the channel upgrade module. USFDA and other regulatory requirements may limit the types of therapy that can be provided simultaneously through the system, but there are no technical reasons why several different therapies cannot be provided at the same time using the invention.

FIG. 14 illustrates four types of therapeutic applicators, each of which is adapted for applying a form of energy to a patient. As shown therein, ultrasound applicator 184 is adapted to deliver high-frequency acoustic vibrations (or ultrasound energy) for therapeutic application to a patient. Applicator 186 is adapted to deliver electromagnetic radiation within a specific range (such as LLLT at a specific wavelength or radio frequency radiation) for therapeutic application to a patient. Adhesive electrodes 188 and vacuum electrodes 190 are both adapted to deliver electrical current to a patient for therapeutic purposes.

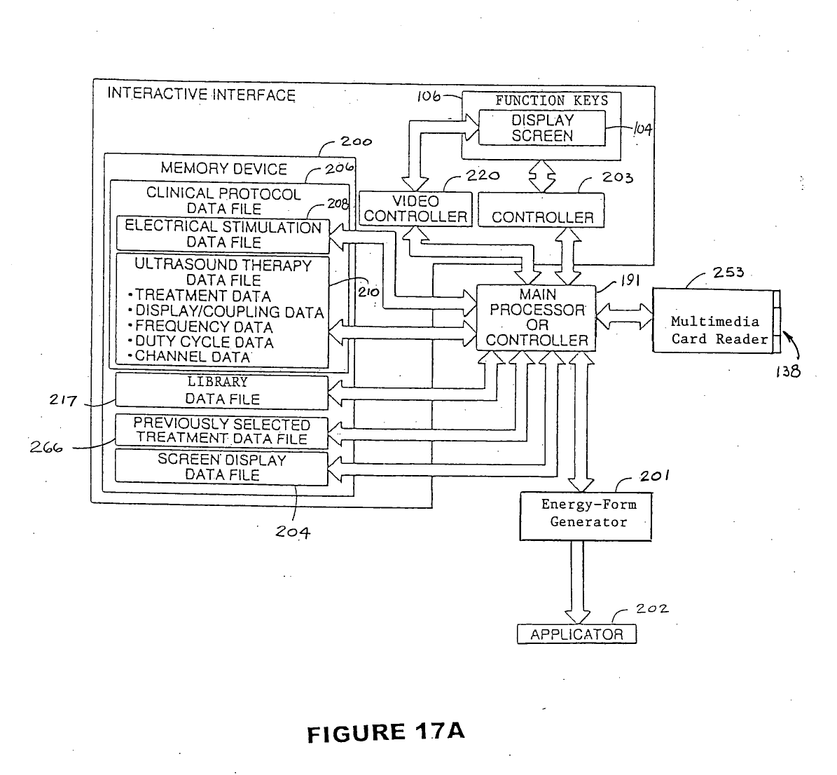

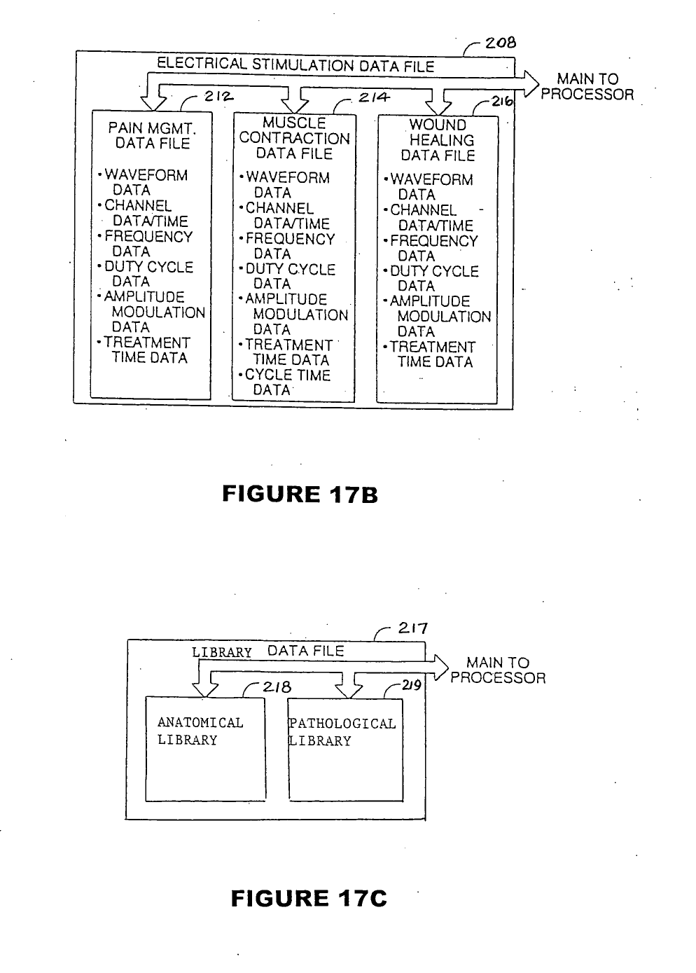

Those having ordinary skill in the art to which the invention relates will understand that the primary unit includes a main processor or controller 191 (shown in FIG. 17A) that is adapted to control delivery of electrical stimulation and ultrasound therapies according to the invention. This controller is also adapted to control the operation of serial devices that are known to those having ordinary skill in the art to which the invention relates, such as an electronic hand dynamometry device, an electrogoniometry device and a pressure pad transducer. In addition, the controller of the primary unit is adapted to be in communication, through the first and second module receivers, with an operating system for each upgrade module, so as to permit control of the upgrade functions through the primary unit. The operation of controller 191 is preferably controlled by a stored programming code. In one embodiment, the programming code preferably is written in C⁺⁺ programming language. The operations of the main controller, however, can be controlled by other types of programming code and languages known to those skilled in the art without departing from the spirit and scope of the present invention.

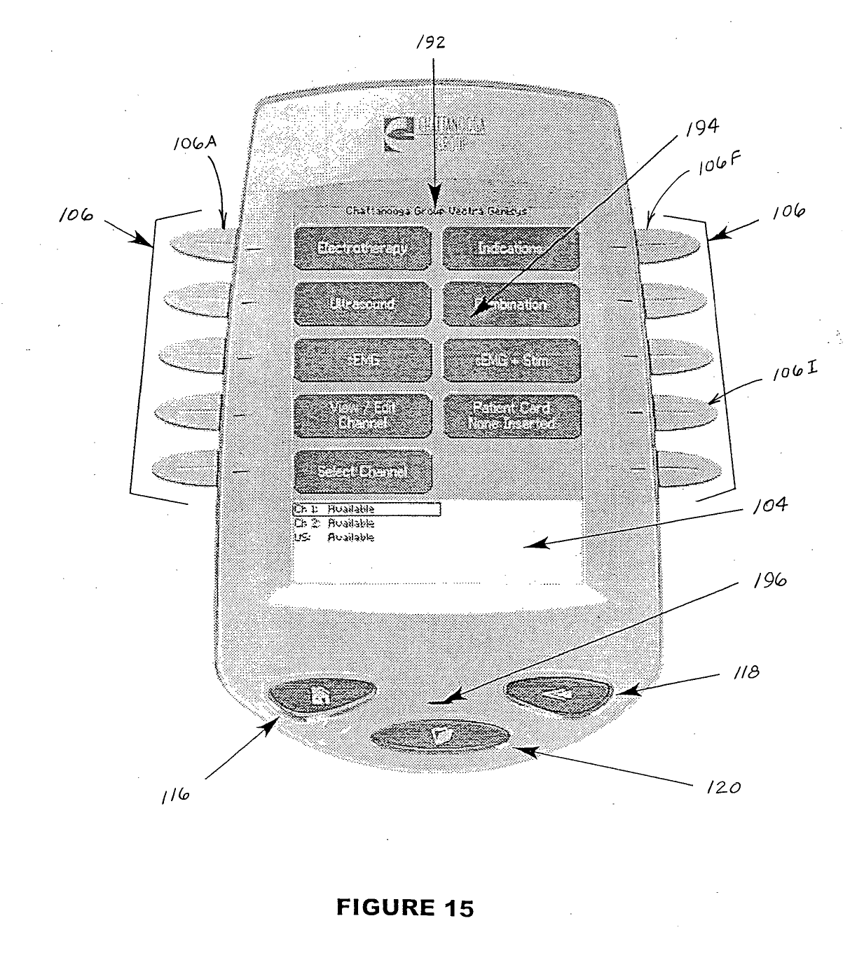

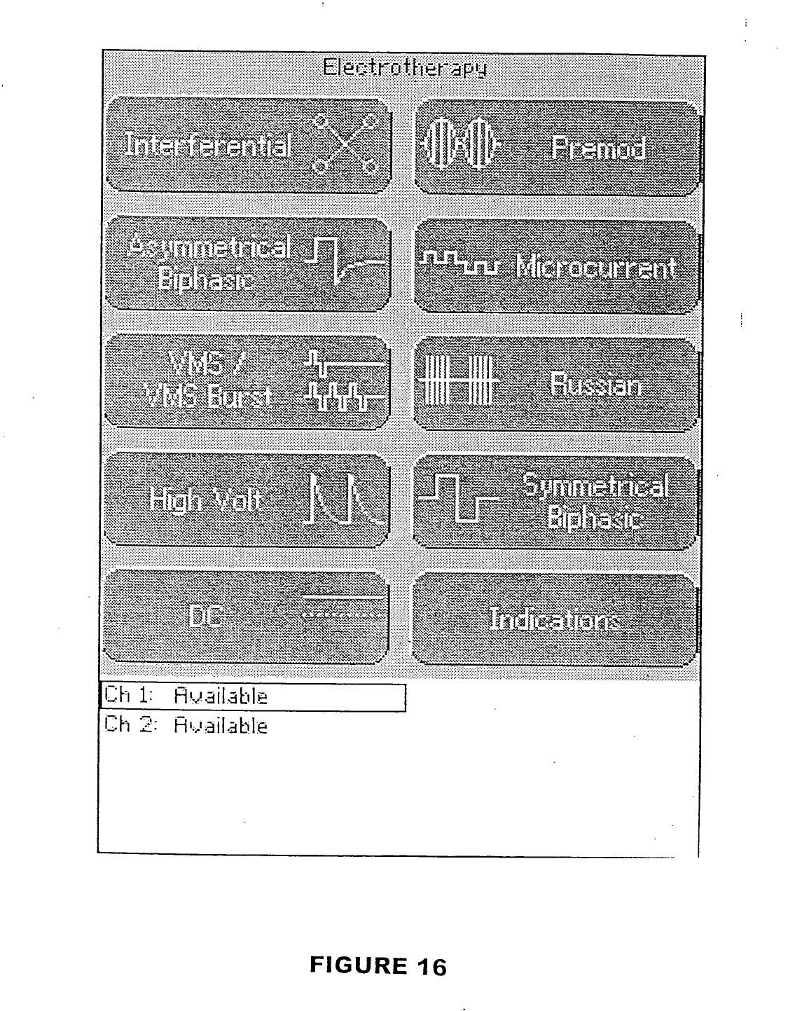

The preferred embodiment of the invention comprises a user interface which includes all of the functions and controls necessary for the operator to access all operator utilities, modalities and parameters of the system. FIG. 15 illustrates display screen 104 and function keys 106 of primary unit 100 with the system “Home Page” being displayed. As shown therein, the top portion 192 of the screen shows the title bar which displays the clinic name (on the Home Page) or which displays other information such as the waveform or modality being used when other pages are displayed. The center portion 194 of the Home Page displays the available modality options. When it is desired to access one of these options, the operator presses the adjacent key 106, such as key 106A which accesses the “Electrotherapy” modality. When a modality is selected, a new “page” will be displayed, providing additional options and parameters for selection by the operator. For example, FIG. 16 illustrates the available waveforms and parameter editing functions that are displayed according to a preferred embodiment of the invention, when the “Electrotherapy” key is pressed on the “Home Page”. Referring again to FIG. 15, power indicator 196 shows whether the system power is turned “On”. As mentioned above in connection with a description of FIG. 1, button 116 is a “Home Page” key which may be used to go to the Home Page from any other page. Button 118 is a “Back” key that is used to return to the previous page. Button 120 accesses the Clinical Resource Library database.

According to the present invention, primary unit 100 includes an interactive ailment-protocol selection interface that is operatively connected to the energy-form generators of the system. As will be apparent to those skilled in the art to which the invention relates, the interactive ailment-protocol selection interface is preferably implemented by a combination of hardware (including screen 104, controller 191 and the various primary unit function keys) and software. In addition, the various components of the interactive ailment-protocol selection interface described herein preferably communicate via electrical command signals, although other types of communication can be employed without departing from the spirit and scope of the invention.

As illustrated schematically in FIG. 17A, the interactive ailment-protocol selection interface includes an ailment data storage device, including memory device 200, for storing identification data representative of at least one physical ailment for each of a plurality of human body parts, such as an acute cervical sprain having well localized pain. The identification data stored by the ailment storage means also includes corresponding sets of energy-form generator operational parameters associated with each predetermined physical ailment and each predetermined body part and defining respective clinical protocols. As described in detail hereinafter, the sets of energy-form generator operational parameters and, more particularly, the clinical protocols, define the configuration of the appropriate energy-form generator (one of which, energy-form generator 201, is illustrated in FIG. 17A) such that the generator provides a predetermined therapeutic treatment to a patient. Preferably, the set of energy-form generator operational parameters is selected such that the resulting therapeutic treatment provided by the energy-form generator is particularly adapted to treat the physical ailment associated with the set of operational parameters.

The set of energy-form generator operational parameters can include a variety of operational parameters based upon the type of therapeutic treatment to be provided by the system. For example, for a therapeutic applicator 202 adapted to provide electrical stimulation, the sets of energy-form generator operational parameters typically define the waveform type, number of channels, frequency, duty cycle, amplitude modulation, cycle time data and treatment time. Alternatively, for an applicator adapted to provide ultrasonic stimulation, the sets of energy-form generator operational parameters typically define the frequency, number of channels, duty cycle, treatment time and display/coupling data.

While the value of the parameters varies based upon the type of treatment associated therewith, a few exemplary parameter ranges are provided hereinbelow for solely purposes of illustration. For example, the waveform type is generally selected from interferential (IFC) four pole (traditional), interferential (IFC) two pole (premodulated), asymmetrical biphasic (TENS), symmetrical biphasic (TENS), microcurrent, VMS™, VMS™ Burst, Russian, monophasic high voltage (either positive, negative or alternating) and direct current, and the number of channels is typically one, two or four depending of the desired type of treatment. Other waveform types that may be available include TENS alternating rectangular, TENS monophasic rectangular, Diadynamic, monophasic rectangular pulsed, monophasic triangular pulsed, galvanic continuous, galvanic interrupted, Träbert (Ultrareiz), surged monophasic rectangular and surged monophasic triangular waveforms.

In addition, the frequency for ultrasonic stimulation is generally between about 1 MHz and about 3.3 MHz, while the frequency for electrical stimulation is typically between about 1 Hz and about 20 KHz. The treatment time also typically varies between about 5 minutes and about 20 minutes, but can be a variety of other lengths of time as known to those skilled in the art. Further, the duty cycle of the stimuli is typically between about 1% and about 20%. As described above, however, each of the parameters can have a variety of other values depending upon the desired type of treatment without departing from the spirit and scope of the present invention.

The ailment data storage device and, in the embodiment illustrated in FIGS. 17A and 17B, the memory device 200 preferably stores the identification data including the sets of energy-form generator operational parameters in a number of different data files. For example, the memory device can store the identification data representative of the displayed information regarding each physical ailment and each body part and, in some embodiments, information regarding the respective clinical protocol suggested for the displayed physical ailment in a screen display data file 204. In addition, the memory device can store the plurality of sets of energy-form generator operational parameters in a clinical protocol data file 206.

As further illustrated in FIG. 17A, the clinical protocol data file 206 can be divided into an electrical stimulation data file 208 and an ultrasound therapy data file 210. Of course, if another energy-form generator were added by upgrade module, such as for example, an LLLT module, the upgrade module would also include a data file for the therapeutic application of the energy-form added. The electrical stimulation data file and the ultrasound therapy data file include the clinical protocols and, more specifically, the sets of energy-form generator operational parameters which configure the generators to provide electrical stimulation and ultrasound therapy, respectively. As illustrated in FIG. 17B, the electrical stimulation data file can be further subdivided based upon the type of electrical stimulation for which the sets of energy-form generator operational parameters configure the energy-form generator (or transducer). For example, the electrical stimulation data file can be subdivided into a pain management data file 212, a muscle contraction data file 214 and a wound healing data file 216. The pain management data file, the muscle contraction data file and the wound healing data file include sets of energy-form generator (or transducer) operational parameters which configure the transducer to deliver electrical stimulation which provides pain management, muscle contraction and wound healing, respectively.

Referring again to FIGS. 17A and 17B, the sets of operational parameters stored in the clinical protocol data file 206 typically include the parameters for configuring energy-form generator 201, as described above. The clinical protocol data file can also include alternative sets of energy-form generator operational parameters for a specific type of therapy to permit an operator more flexibility in providing a desired therapeutic treatment. FIG. 17C illustrates the library data file 217 (also shown in FIG. 17A). The library data file is preferably subdivided into anatomical library 218 containing diagrams and information about body parts, and pathological library 219 containing diagrams and information about physical ailments and pathological conditions.

The interactive ailment-protocol selection interface of the preferred embodiment of the invention includes video controller 220 (illustrated schematically in FIG. 17A) which is associated with display screen 104. In one embodiment, the display screen is a VGA liquid crystal display screen. The display screen, however, can be of another type known to those having ordinary skill in the art to which the invention relates without departing from the spirit and scope of the present invention. As explained in detail hereinafter, the display screen will display at least one of the identification data representative of a plurality of physical ailments which are associated with at least one of the identified human body parts selected by use of the function keys 106.

In the preferred embodiment illustrated in FIG. 15, a main menu is initially displayed on the Home Page of display screen 104. This main menu includes various identification data that prompt the operator to select a desired feature by pressing the adjacent key. For example, the selections can include electrotherapy, ultrasound, a combination of electrotherapy and ultrasound therapy, surface EMG and surface EMG plus electrical stimulation.

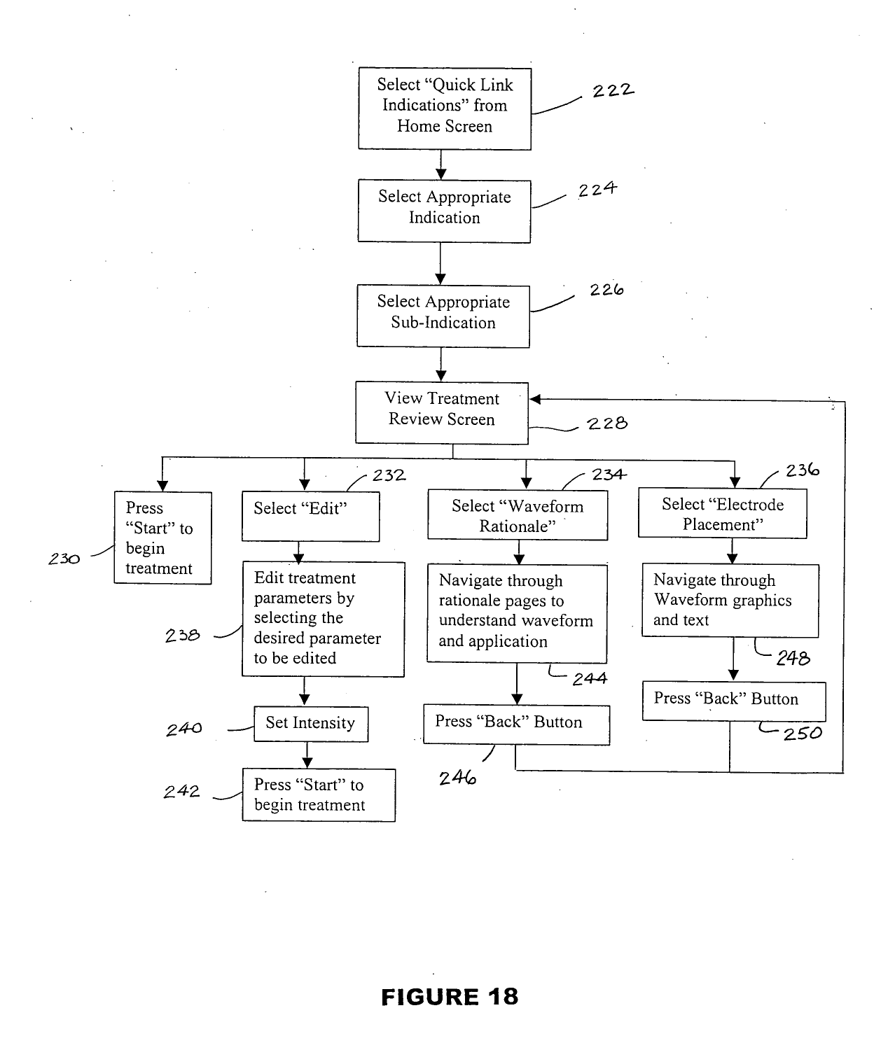

One of the identification data on the main menu page is labeled “Indications” to provide access to this component of a preferred embodiment of the invention. By pressing key 106F, an operator can select a quick link identified as “Indications” (shown as box 222 on FIG. 18). This will produce another page in which the operator could select pain management, muscle contraction or wound healing, for example, as shown in box 224. The following page would then include other identification data representative of subsets of each of these indications. Selection of the appropriate sub-indication, as shown in box 226, would then produce a “Treatment Review” page, as shown in box 228, which includes identification data representative of a “Start” button (shown at 230), an “Edit” feature (shown at 232), a “Waveform Rationale” (shown at 234) or an “Electrode Placement” (shown at 236). At any time during the sequence, Back button 118 may be pressed on the primary unit to go back to the previously displayed page.

If the key adjacent to the “Start” identification datum is pressed, treatment will be initiated just as if the “Start” button 110 on the primary unit were depressed. If the key adjacent to the “Edit” identification datum is pressed, a page of energy-form generator operational parameters associated with the selected indication and sub-indication will be displayed. Any of these parameters can then be edited by selection of appropriate identification data using the adjacent function keys and a protocol modifier comprised of controller 191 and appropriate software. Once the parameters have been edited (or if no edits are desired), a “Next Page” indicator will lead to a page (shown at 240) where the intensity of the energy form to be applied may be set. Of course, the intensity may also be set by turning Intensity button 108 on the primary unit. Once the intensity is set, an identification datum will allow the operator to initiate the treatment according to the selected operational parameters (shown at 242).

Returning now to the Treatment Review page, the waveform rationale for any of the available waveforms may be selected by depressing the appropriate key. This will produce one or more successive pages (shown at 244) for each waveform which describe the rationale behind the use of the waveform for the specific indication selected. After an operator consults the selected waveform rationale, an identification datum may be selected (at 246) to return to the Treatment Review page. A key for selection of the suggested electrode placement for the specific indication selected may then be depressed to produce one or more graphical and textual pages (at 248) which preferably include one or more diagrams showing suggested electrode locations, as well as recommended electrode sizes. Once this information has been consulted, an identification datum may be selected (at 250) to return to the Treatment Review page.

In order to properly configure energy-form generator 201 of the physical therapy applicator 202, the interactive ailment-protocol selection interface also includes a protocol selector that is in electrical communication with the ailment data storage device and responsive to operator selection of identification data representative of a specific physical ailment. Based upon the operator selection, the protocol selector obtains the set of energy-form generator operational parameters associated with the selected physical ailment so as to thereby select the clinical protocol according to which the energy-form generator will be configured.

In the preferred embodiment illustrated in FIGS. 1 and 17A, the protocol selector comprises clinical resources button 120 and function keys 106 which are located adjacent to display screen 104. In the illustrated embodiment, the protocol selector also includes a function key controller 203 which is responsive to function keys 106 for creating the X and Y coordinates which correspond to the display screen locations adjacent to each key. In addition, the protocol selector can include main controller 191 for determining the selection made by an operator who has depressed one of the function keys, based upon the X and Y coordinates generated by controller 203. Although a protocol selector which includes function keys 106 and an associated controller 203 is illustrated and described herein, the specific physical ailment can be selected in a variety of other manners without departing from the spirit and scope of the present invention. For example, the interactive ailment-protocol selection interface can include a keypad, a mouse, a light pen, a touch screen or other selection means known to those skilled in the art for selecting one of the physical ailments displayed by the display screen 104.

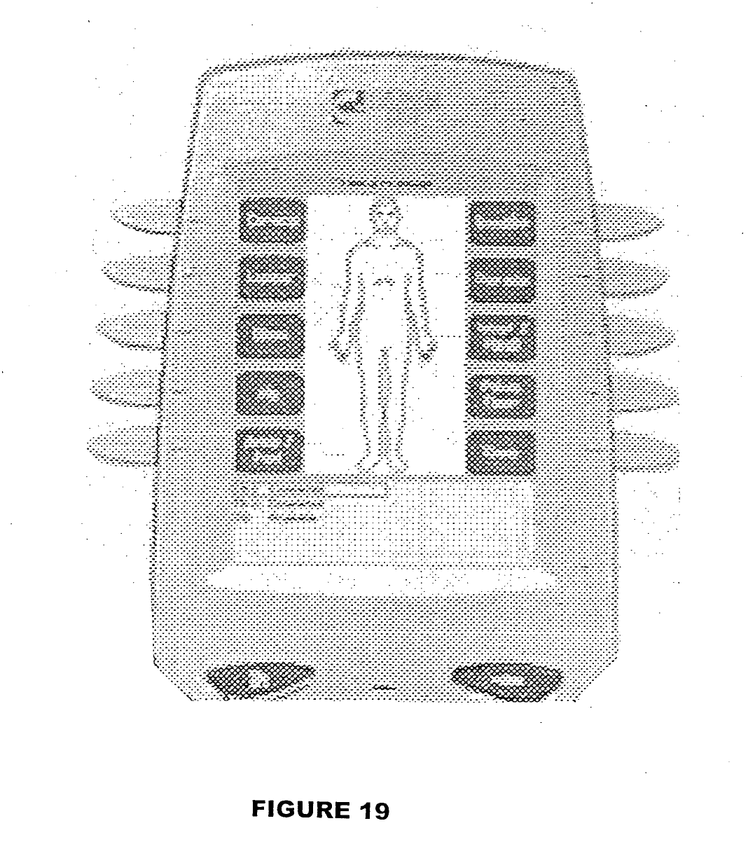

When an operator presses Clinical Resources button 120, the next page displayed will show the identification datum “Clinical Protocols” next to key 106A. Pressing this key will bring up the page illustrated in FIG. 19, which includes a library data selector comprising a basic representation of the human body and a series of identification data representative of various body parts, such as “neck”, “shoulder”, “arm”, “leg”, and “ankle/foot”. Pressing the key adjacent to the selected body part will produce a page listing identification data representative of a plurality of physical ailments which are associated with the previously selected human body part. Successive pages will display identification data representative of the severity of a selected physical ailment, a recommended treatment protocol and recommendations for proper placement of the therapeutic applicator for the desired treatment. When a particular protocol is selected, main controller 191 obtains the set of energy-form generator operational parameters associated with the selected physical ailment from the ailment data storage device. The operator may also edit the parameters for a selected clinical protocol by pressing an “Edit” key on a page and following instructions on the page or pages following (in a manner similar to that described above in connection with the “Indications” component of the preferred embodiment).

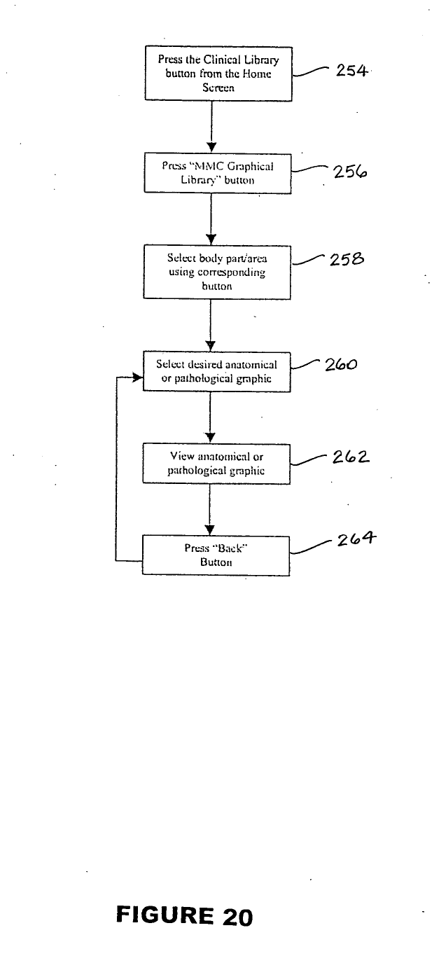

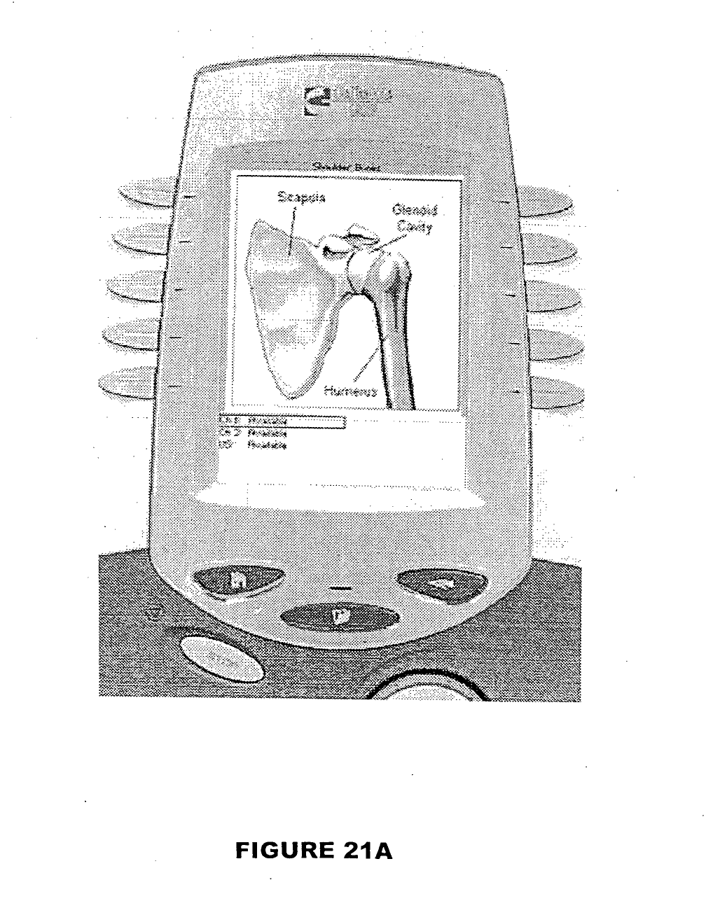

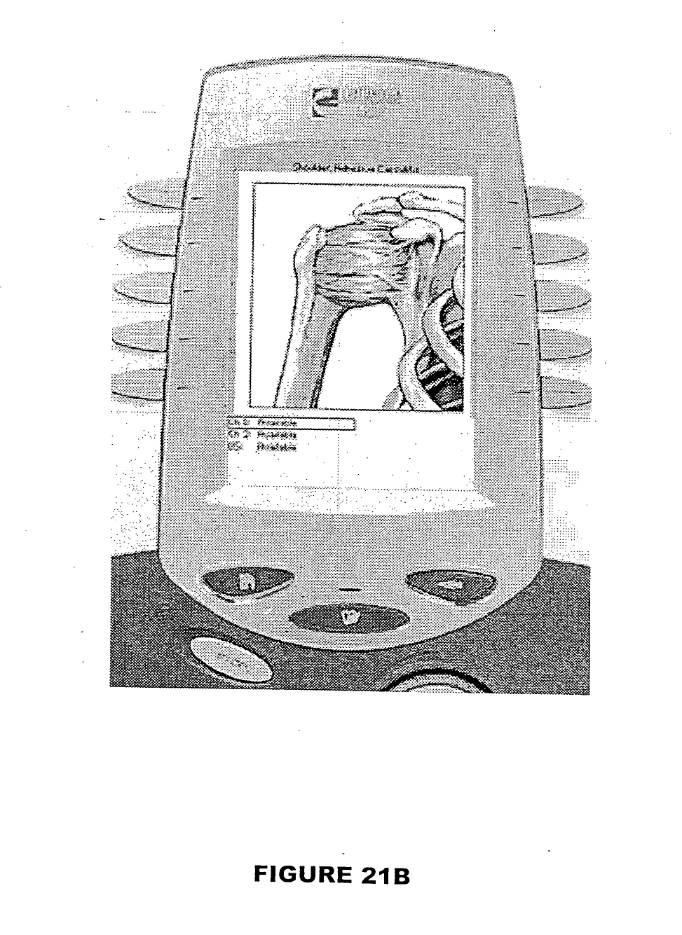

In a preferred embodiment of the invention, more detailed anatomical and pathological information may be obtained inserting a multimedia card 252 (shown in FIG. 12), on which additional information is stored, into multimedia card access port 138 (shown in FIGS. 3 and 17A) of the primary unit. The multimedia card access port is operatively connected to multimedia card reader 253 which is operatively connected to main controller 191. The multimedia card is a data storage device of a conventional type that is known to those having ordinary skill in the art to which the invention relates. FIG. 20 is a flow chart illustrating the use of on-board multimedia card reader 253 to view graphical anatomical and/or pathological libraries on a display screen for a selected body part or area. When an operator inserts multimedia card 252 into card reader 253 through access port 138, the information contained on the card may be accessed by pressing Clinical Resources button 120 on the primary unit (shown at 254 on FIG. 20) and then selecting the identification datum for the Multimedia Library that is displayed on the following page (shown at 256). This will produce a page similar to that shown in FIG. 19 which includes identification data representative of various body parts. When a desired body part is selected by pressing the key adjacent to the appropriate identification datum (shown at 258), a page will be displayed that offers the operator a choice between anatomical and pathological graphic information (shown at 260). The operator may then choose (shown at 262) to obtain anatomical information, an example of which (for the shoulder) is illustrated in FIG. 20A, or the operator may choose pathological information, an example of which is illustrated in FIG. 20B. From either the anatomical or the pathological library, the operator may depress Back button 118 to return to the library selection page (shown at 264).

Referring again to FIG. 16, an operator may also select a particular waveform for application of electrical stimulation by pressing the key (not shown in FIG. 16) adjacent the selected waveform on the “Electrotherapy” page. The next page that appears (not shown) will include identification data associated with a number of more detailed parameters which may be accessed by pressing the adjacent key. The operator may also edit the parameters for a selected waveform by pressing an “Edit” key on a page and following instructions on the following page.

Memory device 200 also includes data file 266 in which a menu of previously selected sets of energy-form generator operational parameters may be saved so that an operator can rapidly reconfigure the energy-form generator according to a previously selected set of operational parameters. These “User Protocols” may be accessed by pressing the Clinical Resources button 120. The next page displayed will show the identification datum “User Protocols” next to key 106F. Pressing this key will bring up the previously saved user protocols. This function is particularly useful in instances in which the energy-form generator is repeatedly configured according to the same set of operational parameters. For example, a physical therapy patient may receive the same treatment every week. Accordingly, the set of energy-form generator operational parameters which configure the energy-form generator to provide this treatment can be stored, such as in previously selected treatment data file 266 in memory device 20.

The preferred embodiment of the primary unit also includes a generator protocol operational mechanism, typically including the main controller or processor 191, which is in electrical communication with generator 201 and is responsive to the protocol selector. The generator protocol operational mechanism configures the energy-form generator to provide therapeutic treatment to the identified body part according to the obtained set of energy-form generator operational parameters. In one embodiment, the generator protocol operational mechanism configures the energy-form generator by providing predetermined signals to the generator indicative of the stimuli to be produced thereby. As understood by those skilled in the art, various techniques of implementing the operational and driving characteristics, i.e., oscillators, power amplifiers, transformers, analog-to-digital computers, of the energy-form generator may be implemented to responsively operate the energy-form generator from the main controller. Accordingly, the energy-form generator can provide a therapeutic treatment which is tailored to specifically treat the selected physical ailment. An operator of primary unit 100 can readily configure the energy-form generator associated with therapeutic applicator 202 based upon the specific physical ailment from which the patient suffers.

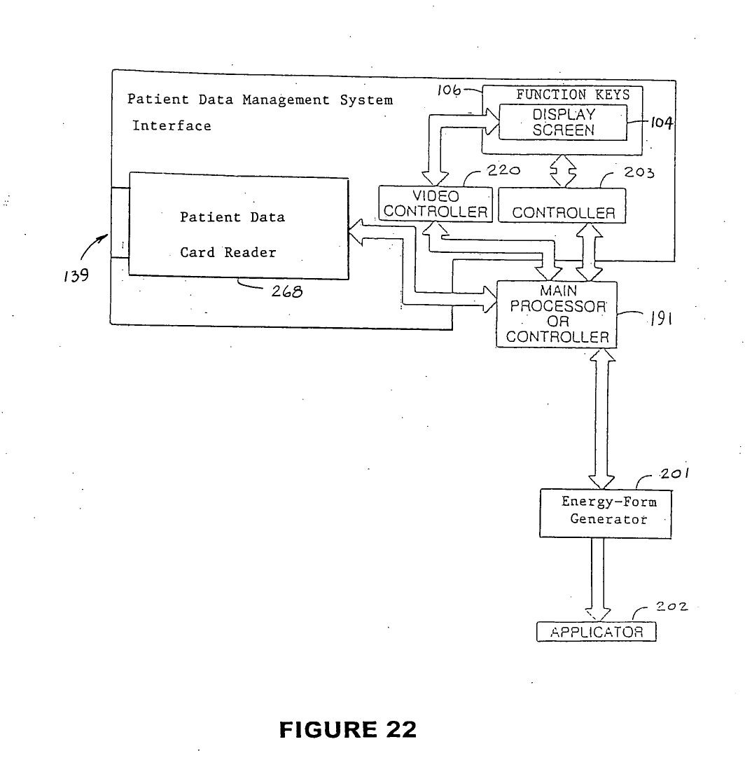

In a preferred embodiment of the invention, primary unit 100 includes a patient data management system (PDMS) interface which is illustrated in FIG. 22. As shown therein, the PDMS interface includes a patient data storage device such as removeable patient data card 176 (shown in FIG. 12) which is adapted to be inserted into patient data card access port 139 (also shown in FIG. 3) into internal card reader 268. In the alternative, an internal memory device similar to memory device 200 of the interactive interface illustrated in FIG. 17A may be provided, along with an internal PDMS data file similar to clinical protocol data file 206 of the embodiment of FIG. 17A.

The patient data storage device is adapted for storing identification data representative of at least one patient and a corresponding set of therapy information associated with each such patient in a file contained in data card 176. The identification data for each patient may include the patient's identification number, name, date of birth or age, address, date of initial consultation and health insurance information. The set of therapy information for each patient can include all therapy session parameters, such as energy-form generator operational parameters for configuring energy-form generator 201, the type and placement of applicator 202, an identification of the patient's pain location and severity of pain reported (based upon a numeric scale and/or a graphical bar scale), as well as operator observations and treatment session notes for each treatment session.

The interactive PDMS interface of the preferred embodiment of the invention includes a patient data input device that is operatively connected to the patient data storage device and to display screen 104. Preferably, this data input device comprises function keys 106, controller 203 and main controller 191. The PDMS interface also includes video controller 220 which is associated with display screen 104. Referring again to FIG. 15, it can be seen that key 106I corresponds to the PDMS identification datum on the system Home Page. When key 106I is selected, a page will follow which offers a selection of patient data card setup or patient data card use. If patient data card setup is selected by use of the appropriate function key, a page showing alphanumeric characters arranged in rows between the function keys may be displayed. Instructions will also appear on this page for selecting alphanumeric characters to enter the patient's identification data. This page will also include an indicator to save the entered information to the patient data card. Other pages can be accessed in a manner similar to that described above in connection with the operation of the system to input therapy information and to save it to the patient data card. In one embodiment of the invention in which an EMG module is employed in the patient's therapy, EMG information may also be saved to a patient data card. The patient data card or cards for each patient may be stored in the patient's file between treatment sessions.

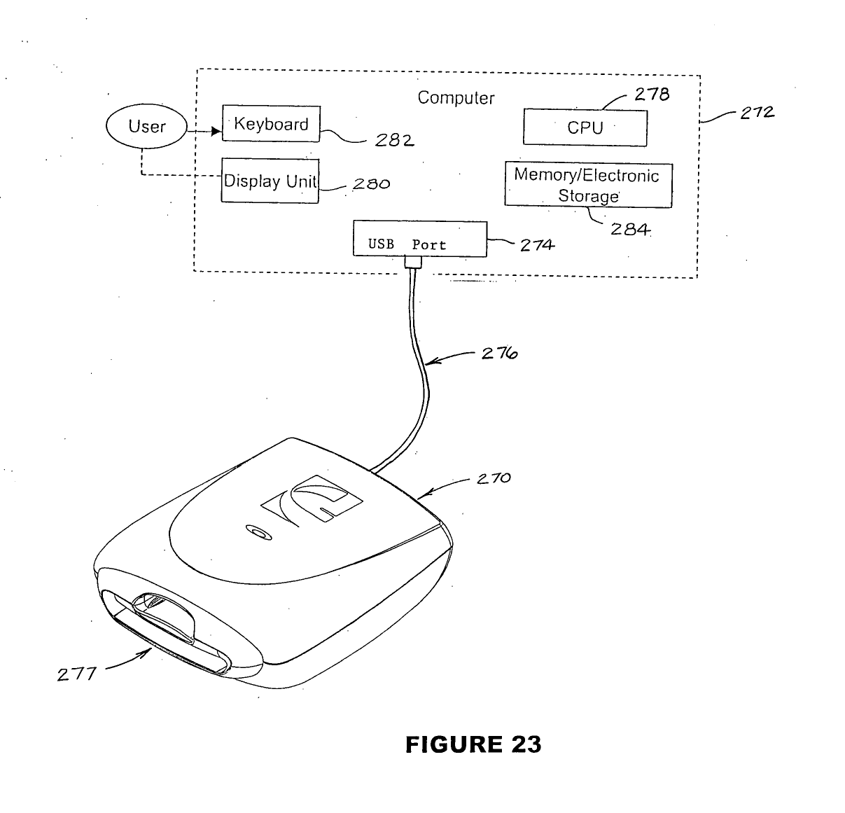

In a preferred embodiment of the invention, the system includes an external read/write device that is operatively connected to a personal computer. Preferred external read/write device 270 (not shown to scale) is illustrated in FIG. 23, along with computer 272. Device 270 is adapted to be connected to the USB port 274 of the computer by cable 276, and it includes a patient data card access port 277 that is adapted to operatively receive a patient data card 176. In this regard, access port 277 is similar to access port 139 of primary unit 100. Computer 272 is preferably a conventional computer which includes central processing unit 278, a display device 280 such as a cathode ray tube (CRT) or liquid crystal display (LCD) monitor, an input device such as keyboard 282, including alphanumeric and other keys (including cursor control keys), and/or a mouse, trackball or similar device. Computer 272 also includes a memory or electronic storage device 284 which includes data and instructions for carrying out various functions, including instructions for the operation of external device 270. The instructions stored in the storage device may be operated by the central processing unit, in cooperation with input device 282 and interactive display unit 280 to permit the input and storage of patient data and associated therapy data to the patient data card. Computer 272 may also be provided with instructions for selecting and organizing information or data from the patient data card and printing this selected information using a printer (not shown). Computer 272 may also be associated with a modem (also not shown) or other input/output device for transmitting information from the patient data card and/or for receiving information from another computer (not shown) and storing it on the patient data card.

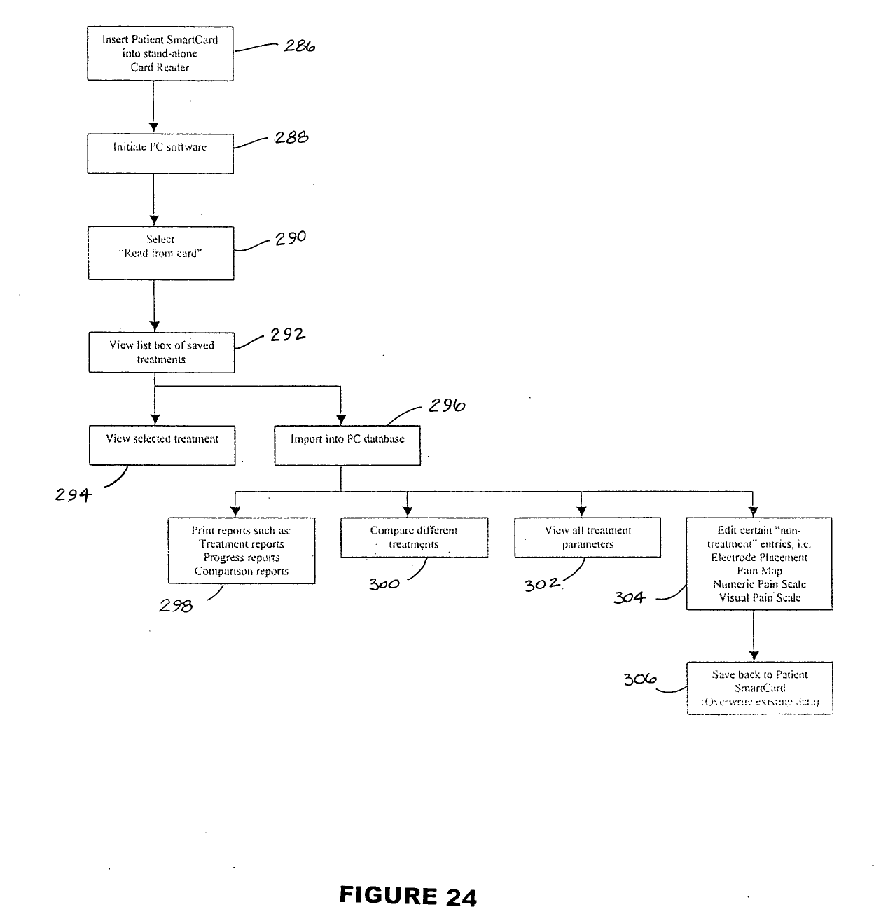

FIG. 24 illustrates the use of external read/write device 270 and personal computer 272 in connection with a preferred embodiment of the invention. As shown therein, patient data card 176 for a patient is inserted into access port 277 (shown at 286), and the software of computer 272 is initiated (shown at 288). Preferably, display unit 280 of computer will display a prompt to read information from the card (shown at 290). A list of identification data for previously saved therapy information about the patient will be displayed and the operator will be prompted select one or more items of therapy information from the list at 292. The detailed therapy information may be viewed (shown at 294) or the therapy information may be imported into memory 284 (shown at 296). Once the information resides in memory 284, reports may be printed (shown at 298), information from different treatment sessions may be compared (shown at 300), all treatment parameters may be viewed (shown at 302) or the information may be edited (shown at 304). If the information is edited, the revised therapy information may then be saved to the patient data card (shown at 306).

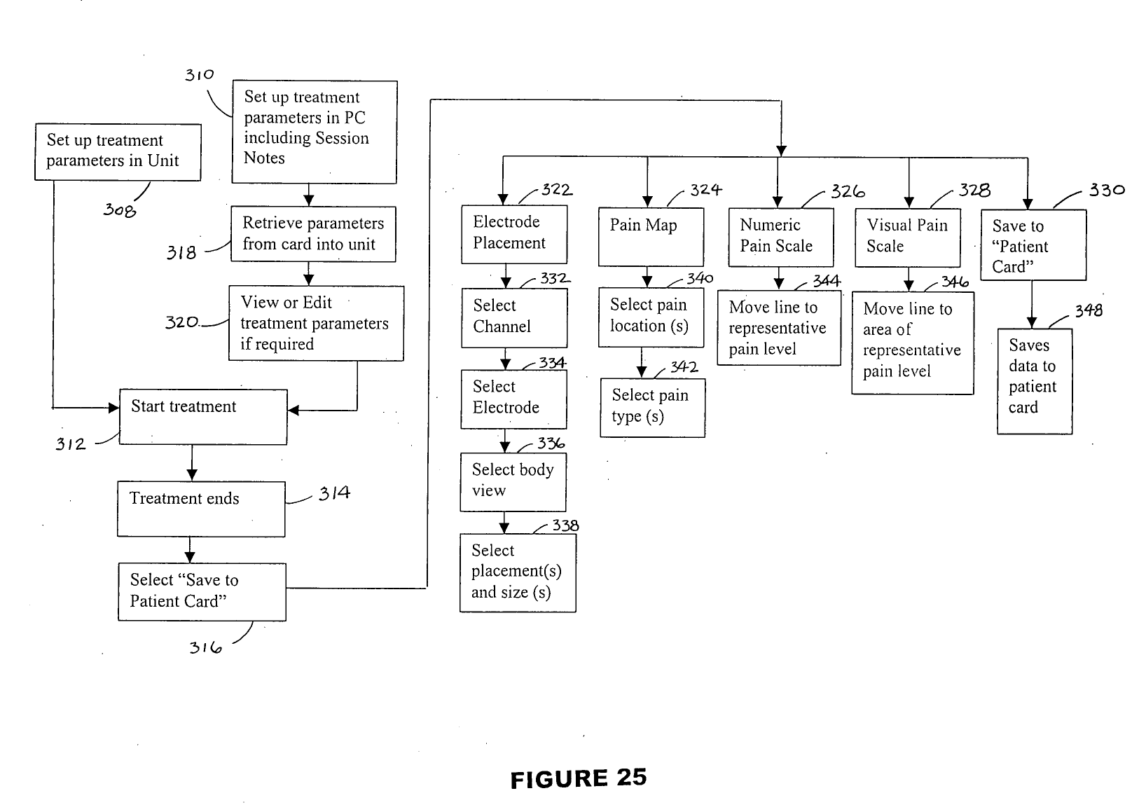

Referring now to FIGS. 22-25, the use of patient data card 176 and internal card reader 268 of a preferred embodiment of the invention will be described. As shown in FIG. 25 and as described herein, the treatment parameters for a patient may be entered into a patient data card using either primary unit 100 (shown at 308) or computer 272 (shown at 310). If the treatment parameters were entered using primary unit 100, FIG. 15 illustrates that key 106I corresponds to the PDMS identification datum on the system Home Page. When key 106I is selected, a page will follow which offers a selection of patient data card Setup or patient data card Use. If patient data card use selected, a page will be displayed offering a selection between “View Treatment Parameters” and “Start Treatment”. If “Start Treatment” is selected at 312, the treatment will proceed according to the parameters stored on card 176 until it ends at 314. If, on the other hand, the card was set up using computer 272, the treatment information must be retrieved by patient data card reader 268 (shown at 318). The treatment information included within this information may be viewed and/or edited at 320 and treatment initiated at 312. The treatment will then proceed according to the parameters stored on the card until it ends at 314.

The information about the treatment session just concluded may then be saved onto the card by selecting “Save To Card” from display screen 104 using an appropriate function key. A page will then be displayed which references several types of information that may be saved to the card, including electrode placement information (at 322), pain map or pain location information (at 324), and information about the reported pain, either based on a numeric scale (at 326) or a graphical bar scale (at 328). A “Save To Card” option (at 330) may also be selected from this page. If electrode placement was selected, successive pages will follow in which the operator can specify or select channels of stimulation used (at 332), types of therapeutic applicator or electrodes used (at 334), the body location from the library where the therapy was applied (at 336) and the location or locations at which the therapeutic applicator or electrodes were applied, and if electrodes were used, the size of the electrodes (at 338). If the pain map was selected, successive pages will follow in which the operator can specify or select the pain location on the body (at 340) and the type of pain experienced (at 342). If the numeric pain scale was selected, a page will follow in which the operator can specify or select the degree of pain experienced (at 344). If the graphical or visual pain scale was selected, a page will follow in which the operator can specify or select the degree of pain experienced (at 346). Finally, if the “Save To Card” option was selected, a page will follow which will permit the new therapy information to be saved to the patient data card.

Although this description contains many specifics, these should not be construed as limiting the scope of the invention but as merely providing illustrations of some of the presently preferred embodiments thereof, as well as the best mode contemplated by the inventors of carrying out the invention. The invention, as described herein, is susceptible to various modifications and adaptations as would be understood by those having ordinary skill in the art to which the invention relates, and the same are intended to be comprehended within the meaning and range of equivalents of the appended claims.

1. A system for the therapeutic application of energy to a patient, said system comprising:
(a) a primary unit which includes:
(i) a first generator for generating a first form of energy for therapeutic application to a patient;
(ii) a first outlet jack that is operatively connected to the first generator and adapted to receive a first therapeutic applicator;
(iii) a module receiver for receiving an upgrade module for enhancing the operation of the system;
(b) a first therapeutic applicator for use in applying the first form of energy to a patient, which applicator is adapted to be operatively connected to the first generator through the first outlet jack.

2. The system of claim 1:
(a) wherein the module receiver is adapted to receive an upgrade module comprising a battery module which is adapted to power the system;
(b) which includes an upgrade module comprising a rechargeable battery which is adapted to be operatively connected to the first generator through the module receiver.

3. The system of claim 1:
(a) wherein the module receiver is adapted to receive an upgrade module comprising an EMG module which is adapted to monitor the extracellular activity of skeletal muscles;
(b) which includes an upgrade module comprising an EMG module which is adapted to be operatively connected to the module receiver for monitoring the extracellular activity of skeletal muscles.

4. The system of claim 1 wherein the first generator is adapted to generate ultrasound energy for therapeutic application to a patient.

5. The system of claim 1 which includes:
(a) a serial connector outlet jack for connection of a serial device which is adapted to enhance the operation of the system;
(b) control means for controlling the operation of the serial device;
(c) a serial device which is adapted to enhance the operation of the system.

6. The system of claim 5 which includes a serial device selected from the group consisting of a hand dynamometry device, an electrogoniometry device and a pressure pad transducer.

7. The system of claim 1 wherein the first generator is adapted to generate electrical current for therapeutic application to a patient.

8. The system of claim 7:
(a) wherein the module receiver is adapted to receive an upgrade module comprising a vacuum module, said vacuum module:
(i) being adapted to be operatively connected to the first generator;
(ii) including a vacuum jack that is adapted to operatively connect a first therapeutic applicator comprising a vacuum electrode to the first generator;
(b) which includes an upgrade module comprising a vacuum module which is adapted to be operatively connected to the module receiver.

9. The system of claim 7:
(a) wherein the first outlet jack is operatively connected to the first generator and adapted to receive a pair of electrodes for use in applying electrical current having a first intensity;
(b) wherein the module receiver is adapted to receive an upgrade module comprising a channel upgrade module, said channel upgrade module:
(i) including a second generator that is adapted to generate electrical current having a second intensity;
(ii) including a second outlet jack that is adapted to operatively connect a pair of electrodes to the second generator;
(c) which includes an upgrade module comprising a channel upgrade module which is adapted to be operatively connected to the module receiver.

10. The system of claim 9 wherein the channel upgrade module includes a secondary module receiver that is adapted to receive a secondary upgrade module for enhancing the operation of the system.

11. The system of claim 10 wherein the secondary module is selected from the group consisting of:
(a) an EMG module which is adapted to monitor the extracellular activity of skeletal muscles;
(b) an energy application module including a second energy-form generator for generating a second form of energy for therapeutic application to a patient.

12. The system of claim 7 wherein the primary unit includes a secondary module receiver that is adapted to receive a secondary upgrade module for enhancing the operation of the system.

13. The system of claim 12 wherein the secondary module is selected from the group consisting of:
(a) an EMG module which is adapted to monitor the extracellular activity of skeletal muscles;
(b) a vacuum module which:
(i) is adapted to be operatively connected to the first generator;
(ii) includes a vacuum jack that is adapted to operatively connect a first therapeutic applicator comprising a vacuum electrode to the first generator; and
(c) a module including a second energy-form generator for generating a second form of energy for therapeutic application to a patient.

14. The system of claim 1:
(a) wherein the module receiver is adapted to receive an upgrade module comprising a second energy-form generator for generating a second form of energy for therapeutic application to a patient;
(b) which includes an upgrade module comprising:
(i) a second energy-form generator for generating a second form of energy for therapeutic application to a patient;
(ii) a second outlet jack that is operatively connected to the second energy-form generator and adapted to receive a second therapeutic applicator;
(c) a second therapeutic applicator for use in applying the second form of energy to a patient, which applicator is adapted to be electrically connected to the second energy-form generator through the second outlet jack.

15. The system of claim 14 wherein the upgrade module is adapted to generate ultrasound energy for therapeutic application.

16. The system of claim 14 wherein the upgrade module is adapted to generate electromagnetic radiation within a range selected from the radio frequency range, the microwave range, the infrared range, the visible light range and the ultraviolet range for therapeutic application.

17. The system of claim 14 wherein the upgrade module includes a secondary module receiver that is adapted to receive a secondary upgrade module for enhancing the operation of the system.

18. The system of claim 17 which includes a secondary upgrade module that is selected from the group consisting of:
(a) a battery module which is adapted to power the system;
(b) an EMG module which is adapted to monitor the extracellular activity of skeletal muscles;
(c) a vacuum module which:
(i) is adapted to be operatively connected to the first generator;
(ii) includes a vacuum jack that is adapted to operatively connect a first therapeutic applicator comprising a vacuum electrode to the first generator;
(d) an energy application module including a third energy-form generator for generating a third form of energy for therapeutic application to a patient.

19. The system of claim 17 wherein:
(a) the first generator is adapted to generate electrical current for therapeutic application to a patient;
(b) the secondary upgrade module is an EMG module which is adapted to monitor the extracellular activity of skeletal muscles.

20. The system of claim 1 which includes:
(a) a first generator protocol operational mechanism that is operatively connected to the first generator and adapted to configure the operational parameters of the first generator;
(b) an interactive ailment-protocol selection interface that is operatively connected to the first generator protocol operational mechanism, said interactive ailment-protocol selection interface including:
(i) an ailment data storage device for storing identification data representative of at least one predetermined physical ailment for each of a plurality of human body parts and a corresponding set of predetermined first generator operational parameters associated with each predetermined physical ailment and each predetermined body part so as to define a clinical protocol;
(ii) a display screen that is operatively connected to the ailment storage device and adapted to display identification data representative of at least one physical ailment for at least one of the predetermined body parts;
(iii) a protocol selector that is operatively connected to the ailment data storage device and to the first generator protocol operational mechanism, said protocol selector being adapted to obtain the set of predetermined first generator operational parameters associated with the selected physical ailment in response to operator selection of one of the physical ailments for which representative identification data is displayed;
wherein operator selection of one of the physical ailments for which representative identification data is displayed will cause the first generator protocol operational mechanism to configure the operational parameters of the first generator with the predetermined first generator operational parameters associated with the selected physical ailment to provide therapeutic treatment to the selected body part.

21. The system of claim 20 wherein the interactive ailment-protocol selection interface includes a protocol modifier by which an operator may modify any of the predetermined first generator operational parameters associated with a predetermined physical ailment and save the so-modified first generator operational parameters in association with the predetermined physical ailment.

22. The system of claim 1 which includes:
(a) a first generator protocol operational mechanism that is operatively connected to the first generator and adapted to configure the operational parameters of the first generator;
(b) an interactive ailment-protocol selection interface that is operatively connected to the first generator protocol operational mechanism, said interactive ailment-protocol selection interface including:
(i) an ailment data storage device for storing identification data representative of at least one predetermined physical ailment for each of a plurality of human body parts and a corresponding set of first generator operational parameters associated with each predetermined physical ailment and each predetermined body part so as to define a clinical protocol;
(ii) a display screen that is operatively connected to the ailment storage device and adapted to display identification data representative of at least one physical ailment for at least one of the predetermined body parts;
(iii) a protocol selector that is operatively connected to the ailment data storage device and to the first generator protocol operational mechanism, said protocol selector being adapted to permit an operator to input a custom set of first generator operational parameters associated with the selected physical ailment to cause the first generator protocol operational mechanism to configure the operational parameters of the first generator with the custom set of first generator operational parameters associated with the selected physical ailment to provide therapeutic treatment to the selected body part.

23. The system of claim 1 which includes an interactive information selection interface comprising:
(a) a library database storage device for storing predetermined sets of information about a plurality of human body parts and a plurality of physical ailments and a corresponding identification datum for each set of information;
(b) a display screen that is operatively connected to the library database storage device and adapted to display information and identification data contained in such library database storage device;
(c) a library data selector that is operatively connected to the library database storage device and to the screen display, said library data selector being adapted to obtain the predetermined set of information associated with the identification datum corresponding to the selected human body part or the selected physical ailment in response to operator selection of one or the human body parts or physical ailments for which an identification datum is displayed;
wherein operator selection of one of the human body parts or one of the physical ailments for which an identification datum is displayed will cause the screen display to display the information associated with the selected human body part or the selected physical ailment.

24. The system of claim 23 which includes:
(a) a first generator protocol operational mechanism that is operatively connected to the first generator and adapted to configure the operational parameters of the first generator;
(b) an interactive ailment-protocol selection interface that is operatively connected to the first generator protocol operational mechanism, said interactive ailment-protocol selection interface including:
(i) an ailment data storage device for storing identification data representative of at least one predetermined physical ailment for each of a plurality of human body parts and a corresponding set of first generator operational parameters associated with each predetermined physical ailment and each predetermined body part so as to define a clinical protocol;
(ii) a display screen that is operatively connected to the ailment storage device and adapted to display identification data representative of at least one physical ailment for at least one of the predetermined body parts;
(iii) a protocol selector that is operatively connected to the ailment data storage device and to the first generator protocol operational mechanism, said protocol selector being adapted to permit an operator to input a custom set of first generator operational parameters associated with the selected physical ailment to cause the first generator protocol operational mechanism to configure the operational parameters of the first generator with the custom set of first generator operational parameters associated with the selected physical ailment to provide therapeutic treatment to the selected body part.
(c) means for storing identification data representative of a selected human body part or physical ailment in association with a set of first generator operational parameters in the ailment data storage device.

25. The system of claim 1 which includes a patient information storage system comprising:
(a) a patient data storage device for storing identification data representative of at least one patient and a corresponding set of therapy information associated with each such patient;
(b) a display screen that is operatively connected to the patient data storage device and adapted to display therapy information and identification data contained in such patient data storage device;
(c) a patient data input device that is operatively connected to the patient data storage device and the screen display and adapted to input therapy information and identification data for each such patient.

26. The system of claim 25:
(a) wherein the patient data storage device is a removable patient data card;
(b) wherein the patient data input device is adapted to permit an operator to input first generator operational parameters associated with a physical ailment for a patient and to store such first generator operational parameters on the patient data card;
(c) which includes a patient data card reader that is adapted to initiate operation of the first generator utilizing the first generator operational parameters stored on the patient data card.

27. The system of claim 1 which includes a patient information storage system comprising:
(a) a patient data storage device comprising a removable patient data card for storing identification data representative of at least one patient and a corresponding set of therapy information associated with each such patient;
(b) an external read/write device that is adapted to be operatively connected to a personal computer;
(c) instructions for the personal computer to permit the addition of information to the patient data card by or through the personal computer.

28. The system of claim 27:
(a) wherein the personal computer and the external read/write device are adapted to permit an operator to input first generator operational parameters associated with a physical ailment for a patient and to store such first generator operational parameters on the patient data card;
(b) which includes a patient data card reader that is adapted to initiate operation of the first generator utilizing the first generator operational parameters stored on the patient data card.

29. A system for the therapeutic application of energy to a patient, said system comprising:
(a) a primary unit which includes:
(i) a first generator for generating a first form of energy for therapeutic application to a patient;
(ii) a first outlet jack that is operatively connected to the first generator and adapted to receive a first therapeutic applicator;
(b) a first therapeutic applicator for use in applying the first form of energy to a patient, which applicator is adapted to be operatively connected to the first generator through the first outlet jack;
(c) a patient information storage system comprising:
(i) a patient data storage device for storing identification data representative of at least one patient and a corresponding set of therapy information associated with each such patient;
(ii) a display screen that is operatively connected to the patient data storage device and adapted to display therapy information and identification data contained in such patient data storage device;
(iii) a patient data input device that is operatively connected to the patient data storage device and the screen display and adapted to input therapy information and identification data for each such patient.

30. The system of claim 29:
(a) wherein the patient data storage device is a removable patient data card;
(b) wherein the patient data input device is adapted to permit an operator to input first generator operational parameters associated with a physical ailment for a patient and to store such first generator operational parameters on the patient data card;
(c) which includes a patient data card reader that is adapted to initiate operation of the first generator utilizing the first generator operational parameters stored on the patient data card.

31. A system for the therapeutic application of energy to a patient, said system comprising:
(a) a primary unit which includes:
(i) a first generator for generating a first form of energy for therapeutic application to a patient;
(ii) a first outlet jack that is operatively connected to the first generator and adapted to receive a first therapeutic applicator;
(b) a first therapeutic applicator for use in applying the first form of energy to a patient, which applicator is adapted to be operatively connected to the first generator through the first outlet jack;
(c) a patient information storage system comprising:
(i) a patient data storage device comprising a removable patient data card for storing identification data representative of at least one patient and a corresponding set of therapy information associated with each such patient;
(ii) an external read/write device that is adapted to be operatively connected to a personal computer;
(iii) instructions for the personal computer to permit the addition of information to the patient data card by or through the personal computer.

32. The system of claim 31:
(a) wherein the personal computer and the external read/write device are adapted to permit an operator to input first generator operational parameters associated with a physical ailment for a patient and to store such first generator operational parameters on the patient data card;
(b) which includes a patient data card reader that is adapted to initiate operation of the first generator utilizing the first generator operational parameters stored on the patient data card.
